(19) United States
(12) Patent Application Publication
(10) Pub. No.: US 2016/0310664 A1
(43) Pub. Date: Oct. 27, 2016

(54) DRUG INFUSION SYSTEM WITH REUSABLE AND DISPOSABLE COMPONENTS

(71) Applicant: CALIBRA MEDICAL, INC., REDWOOD CITY, CA (US)

(72) Inventors: John MCKENZIE, San Carlos, CA (US); Travis MARSOT, Mountain View, CA (US); Brett CARTER, Redwood City, CA (US); Brett CROSS, Seattle, WA (US)

(21) Appl. No.: 15/137,204

(22) Filed: Apr. 25, 2016

Related U.S. Application Data
(62) Division of application No. 12/543,352, filed on Aug. 18, 2009, now abandoned.
(60) Provisional application No. 61/227,157, filed on Jul. 21, 2009.
(60) Provisional application No. 61/089,749, filed on Aug. 18, 2008.

(51) Int. Cl. A61M 5/142

(52) U.S. Cl. CPC A61M 5/14248; A61M 2005/14252; A61M 2005/14268; A61M 5/1424; A61M 5/1452

(57) ABSTRACT

The application discloses a drug infusion system comprising a base and a drug dispenser. The base is configured to receive a cannula that delivers a drug to beneath a wearer's skin. The base is attachable to the skin of the wearer. The reusable drug dispenser is removably attachable to the base and has a pump unit configured to establish fluid communication between a removably attachable drug reservoir and the cannula. The pump unit pumps the drug to the wearer upon activation by the wearer. The pump unit may have an inlet to contact the drug within the reservoir, which may be a needle. The pump unit may also have a receiving unit to receive the reservoir. Such a receiving unit may be a tubular for a cylindrical reservoir, or may have a cavity or an encasing unit to hold the reservoir.

### PRIORITY CLAIM

The present application claims the benefit of copending U.S. Provisional Patent Application Ser. No. 61/089,749, filed Aug. 18, 2008; the present application also claims the benefit of copending U.S. Provisional Patent Application Ser. No. 61/227,157, filed Jul. 21, 2009; all of the foregoing applications are incorporated herein by reference in their entireties.

### BACKGROUND OF THE INVENTION

The present invention relates to infusion devices and more particularly to such devices that enable liquid medicaments to be conveniently and safely self-administered by a patient. One liquid medicament that is often self-administered by a patient is insulin, and for ease of description, the administration of insulin is generally used herein for exemplary purposes although the invention should not be limited by that exemplary use.

Administration of insulin has traditionally been accomplished using a syringe. Recently, needle carrying pen-like devices have also been employed for this purpose. Both forms of insulin administration require the patients to stick themselves each time they inject insulin, often many times a day. Additionally, a new clean needle must be mounted on the device each time they are used, and disposed of after each use, creating the additional problem of having the “sharps” with them whenever the patient needs to administer insulin, and to safely dispose of them after each use. Thus, these traditional forms of insulin administration have been a rather pervasive intrusion in the lives and routines of the patients who have had to adopt and employ them.

More recently, insulin pumps attached by tubing to an infusion set mounted on the patient's skin have been developed as an alternative form of insulin administration. Such pumps may be controlled by a programmable remote electronic system employing short range radio communication between a control device and electronics that control the pump. While such devices may involve fewer needle sticks, they are expensive to manufacture. They are also complex to operate and cumbersome and awkward to wear. Further, the cost of such devices can be many times the daily expense of using a traditional injection means such as a syringe or an insulin pen.

Devices of the type mentioned above also require a significant amount of training to control and thus use the devices. Great care in programming the devices is required because the pumps generally carry sufficient insulin to last a few days. Improper programming or general operation of the pumps can result in delivery of an excessive amount of insulin which can be very dangerous and even fatal.

Many patients are also reluctant to wear a pump device because they can be socially awkward. The devices are generally quite noticeable and can be as large as a pager. Adding to their awkwardness is their attachment to the outside of the patients clothes and the need for a catheter like tubing set running from the device to an infusion set located on the patient's body. Besides being obvious and perhaps embarrassing, wearing such a device can also be a serious impediment to many activities such as swimming, bathing, athletic activities, and many activities such as sun bathing where portions of the patient's body are necessarily uncovered.

In view of the above, a more cost effective and simple device has been proposed whereby an injection system is discreetly attached directly to the skin of the patient. One example of such a device is described in detail in U.S. application Ser. No. 12/147,283 filed Jun. 26, 2008 and titled DISPOSABLE INFUSION DEVICE WITH REDUNDANT VALVED SAFETY, which application is owned by the assignee of this application and incorporated herein by reference in its entirety. Such a device may be attached to the patient under the patient's clothing to deliver insulin into the patient by the manual pumping of small doses of insulin out the distal end of a temporarily indwelling cannula that is made a part of the pump device. The device may be made quite small and, when worn under the clothes, entirely unnoticeable in most social situations. It may still carry sufficient insulin to last a patient several days. It can be colored to blend naturally with the patient's skin color so as not to be noticeable when the patient's skin is exposed. As a result, insulin for several days may be carried by the patient discreetly, and conveniently applied in small dosages after only a single needle stick. For another description of devices of this type, reference may also be had to co-pending application Ser. No. 11/906,130, filed on Sep. 28, 2007 for DISPOSABLE INFUSION DEVICE WITH DUAL VALVE SYSTEM, which application is owned by the assignee of this application and hereby incorporated herein by reference in its entirety.

Although relatively discrete, the patient may have a reason to remove the system entirely. Likewise, if the drug delivery system is accidentally dislodged from the patient, it would be advantageous to be able to salvage the medicament and pump, and to replace only the minimum amount of the system. Where the pump, insulin supply and cannula are integral and non-separable units, removing just the pump or just the insulin, or adding a different liquid medicament is not generally feasible. Sometimes it would be advantageous to be able to remove the pump unit, the insulin reservoir, or the entire device, and to reassemble and use parts of the drug delivery system. Additionally, since the portion of system that contains the cannula needs to be removed and reinstalled every three days pursuant to current medical and regulatory practice, it would be advantageous to be able to remove the other portions of the drug delivery system from the portion with the cannula, and reattach them to a new cannula containing portion, thus avoiding replacing them with every use.

Further, it would be advantageous if the device was configured to utilize commercially available reservoirs or cartridges. For example, glass cartridges are presently used for insulin injection pens, which are readily available to the patient with a prescription. It would be beneficial to some patients to combine the availability of these cartridges with a discreet device that removes the attendant problems of a syringe-pen. Such a device would also decrease the attendant manufacturing costs of a device that utilizes proprietary reservoirs. More importantly, it would mitigate the inconvenience to the patient of filling or refilling a reservoir and the attendant problems associated with the patient performing that task.

Therefore there is a need for an invention that makes it possible to have a small, simple and discreet drug delivery system and yet be able to remove various components of the drug delivery system from each other, and to reattach them to each other without the need to discard the entire system.

### SUMMARY OF THE INVENTION

In one embodiment, a drug infusion system comprises a base having a cannula well arranged to receive a cannula that conducts the drug to beneath a wearer's skin. The base further includes a base surface arranged to attach to the skin of the wearer. The base includes the cannula well and is arranged to dispose a cannula to extend from the base surface to beneath the wearer's skin. The base further includes an inlet arranged to receive the drug, a conduit that conducts the drug from the inlet to the cannula well, and a first self sealing penetrable barrier moveable with respect to the inlet. The system further includes a reusable drug dispenser removably attachable to the base and having a second self sealing penetrable barrier, a reservoir arranged to hold the drug, and a pump that pumps the drug to the second self sealing penetrable barrier. The first and second self sealing penetrable barriers are arranged to engage each other and to be penetrated by the inlet of the base when the reusable dispenser is attached to the base to form a antiseptic connection between the cannula well and the reservoir.

The inlet may comprise a needle. The system may further comprise a latch assembly that releasably holds the reusable dispenser on the base. The latch assembly may include a male/female clasp arrangement. The clasp arrangement and first and second sealing penetrable barrier may be arranged such that as the male/female clasp arrangement engages, the first and second self sealing penetrable barriers engage each other and are penetrated by the needle to discard the entire system.

The pump may be any one of acceptable drug delivery pumps which may include, for example, a piston pump, a peristaltic pump, a screw pump, a membrane pump, a metering device, and a gas driven positive displacement pump. The system may further comprise a cannula set including a receiving pike and the cannula. The receiving pike may be arranged to be received within the cannula well in fluid communication with the conduit, whereby a fluid connection is formed from the cannula through the conduit to the reservoir. The cannula set may further include a top sealing member. The cannula set may further include a port aligned with the cannula that facilitates placement of the cannula set into the cannula well and a port cover that blocks the port to preclude direct access to the cannula through the port after the cannula set is received within the cannula well. The cannula may be arranged to be deployed beneath the wearer's skin with a drive needle that extends through the port and carries the cannula into the deployed position and the cover may be arranged to block the port upon withdrawal of the needle from the cannula set after deployment of the cannula. The port cover may be formed of resilient material and be arranged to spring over and block the port responsive to the drive needle being withdrawn from the port.

The base may include a guide that guides the reusable dispenser into attachment on the base. The base lower surface may include an adhesive that attaches the base to the wearer's skin. The base may be coextensive with the reusable dispenser when the reusable dispenser is attached to the base. The reusable dispenser may include an inlet cavity adjacent the second self sealing penetrable barrier that receives the inlet of the base when the reusable dispenser is attached to the base. The inlet cavity may be arranged to receive the drug from the reservoir and provide the drug to the inlet of the base. The reusable dispenser may include a pair of actuators operatively associated with the pump for causing the pump to pump the drug form the reservoir to the cannula upon concurrent actuation of the actuators.

The inlet of the base may have a distal end that penetrates the first and second self sealing penetrable barriers when the reusable dispenser is attached to the base. The first self sealing penetrable barrier may be moveable with respect to the inlet of the base and the base may further include a biasing element that urges the first self sealing penetrable barrier against the second self sealing penetrable barrier when the reusable dispenser is attached to the base.

The system may further comprise a vent immediately adjacent the second self sealing penetrable barrier. The vent may comprise a hydrophobic vent covered by a one-way valve that allows the passage of air out the vent, does not allow liquid such as liquid medicament out the vent, and after the pathway is vented, does not allow air back into the fluid pathway.

The reusable portion may comprise a separate reservoir unit that holds the drug to be delivered. The reservoir unit may be engageable with the dispenser such as a pump portion, and both portions may be releasably secured to the base. The reusable portion may further comprise at least one latch that maintains the dispenser and reservoir unit in engagement. The at least one latch may comprise a latching projection and a projection receiving slot.

The latching projection may be carried by the dispenser and the projection receiving slot may be formed in the reservoir unit. The system may further comprise an antiseptic coupling between the dispenser and reservoir unit.

An alternative embodiment for a drug infusion system is also disclosed. The alternative embodiment comprises a base configured to receive a cannula that delivers a drug to beneath a wearer's skin. The base further includes a base surface arranged to attach to the skin of the wearer and is arranged to dispose the cannula to extend from the base surface to beneath the wearer's skin. The device further comprises a reusable drug dispenser removably attachable to the base. The reusable drug dispenser has a pump unit configured to establish fluid communication between a removably attachable drug reservoir and the cannula, whereby the pump unit pumps the drug to the wearer upon activation of the pump by the wearer.

The pump unit may further comprise an inlet configured to contact the drug within the reservoir, and the inlet may be a needle. The pump unit may also comprise a receiving unit configured to receive the reservoir. Such a receiving unit may be tubular to accommodate a cylindrical reservoir. The receiving unit may comprise a cavity configured to hold the reservoir. The pump unit may comprise an encasing unit configured to hold the reservoir, and such an encasing unit may be positioned to one side of the pump unit, thereby allowing a lower profile.

In another embodiment, a drug infusion assembly comprises a base including a base surface arranged to attach to the skin of a wearer. The base includes a cannula arranged to extend from the base surface to beneath the wearer's skin and an inlet in fluid communication with the cannula. The infusion assembly further comprises a pump unit removably attachable to the base. The pump unit has a cavity and a latch assembly within the cavity. The cavity is arranged to receive a cartridge reservoir therein and the latch assembly is arranged to releasably lock the cartridge reservoir within the cavity. The pump unit is configured to establish fluid communication between the releasably locked cartridge reservoir and the inlet of the base and to pump a liquid medicament stored in the cartridge reservoir to the inlet of the base and the cannula upon activation by the wearer.

### BRIEF DESCRIPTION OF THE DRAWINGS

The features of the present invention which are believed to be novel are set forth with particularity in the appended claims. The invention, together with further features and advantages thereof, may best be understood by making reference to the following description taken in conjunction with the accompanying drawings, in the several figures of which like reference numerals identify identical elements, and wherein:

FIG. 1 is a top perspective view of a drug infusion system according to an embodiment of the invention;

FIG. 2 is a bottom plan view of the drug infusion system of FIG. 1;

FIG. 2B is a simplified side view of an alternative embodiment of the device shown in FIGS. 1 and 2;

FIG. 2C is a side view of the embodiment of FIG. 2B during cannula insertion;

FIG. 2D is a side view of the embodiment of FIG. 2B after cannula insertion;

FIG. 3 is a top perspective view of the base portion of the system of FIG. 1;

FIG. 4 is a top perspective view of the base portion of the system of FIG. 1 with a cannula set aligned therewith for deployment;

FIG. 5 is a partial sectional side view, to an enlarged scale, illustrating details of the cannula set and base portion prior to cannula set deployment;

FIG. 6 is a partial sectional side view, to an enlarged scale, illustrating details of the cannula set and base portion after cannula set deployment;

FIG. 7 is a bottom perspective view of the reusable portion of the system of FIG. 1;

FIG. 8 is a top perspective view of the reusable portion being mated with the base portion of the system of FIG. 1;

FIG. 9 is a side view in section, to an enlarged scale, of the antiseptic coupling between the base portion and the reusable portion prior to their engagement;

FIG. 10 is a side view in section, to an enlarged scale, of the antiseptic coupling between the base portion and the reusable portion during the process of their engagement;

FIG. 11 is a side view in section, to an enlarged scale, of the antiseptic coupling between the base portion and the reusable portion after their engagement;

FIG. 12 is a side view in section, to an enlarged scale, of the antiseptic coupling between the base portion and the reusable portion of another drug infusion system according to another embodiment of the invention;

FIG. 13 is a perspective view of another drug infusion system embodying the invention having detachable pump component and reservoir component;

FIG. 14 is a top plan view of the system of FIG. 13 showing reservoir and pump components thereof aligned for engagement;

FIG. 15 is a top plan view of the system of FIG. 13 showing the reservoir and pump components thereof entering engagement; and

FIG. 16 is a top plan view of the system of FIG. 13 showing the reservoir and pump components thereof after engagement.

FIG. 17 is a side view in section of the attachment mechanism between the reservoir portion and pump portion of one embodiment of the invention;

FIG. 18 is a side sectional view to an expanded scale of the connection mechanism between the reservoir portion and the pump portion of one embodiment of the invention;

FIG. 19A is a top view, in perspective, of an embodiment of the invention using a commercially available cartridge;

FIG. 19B is a bottom view of the embodiment of FIG. 19A;

FIG. 19C is a partial side view, in section, of the embodiment of FIG. 19A;

FIG. 20A is a top plan view of another embodiment of the invention using a commercially available cartridge;

FIG. 20B is a side view, in section, of the embodiment of FIG. 20A;

FIG. 20C is a side view of the embodiment of FIG. 20A;

FIG. 20D is a top view of the embodiment of FIG. 20A;

FIG. 21A is a top view of another embodiment Of the invention using a commercially available cartridge;

FIG. 21B is an end view of the embodiment of FIG. 21A;

FIG. 22 is an exploded view of the components of a further infusion assembly embodying the present invention;

FIG. 23 is a perspective view of the assembled components of the assembly of FIG. 22;

FIG. 24 is a bottom view, in perspective, of the assembled components of the assembly of FIG. 22 prior to deployment on a user;

FIG. 25 is a perspective view, with portions cut away, illustrating the releasable lock of the cartridge reservoir within the pump unit;

FIG. 26 is a top plan view, with portions removed, illustrating the cartridge reservoir being loaded into the pump unit and just prior to being releasably locked therein;

FIG. 27 is a top plan view, with portions removed, illustrating the cartridge reservoir after being loaded into the pump unit and being releasably locked therein;

FIG. 28 is an end view, in perspective, of the base unit of the assembly of FIG. 22;

FIG. 29 is a top plan view of the assembled assembly of FIG. 22, with portions removed, illustrating a first condition of a compression spring contacting a cartridge reservoir;

FIG. 30 is a side plan view, with portions removed, of the assembled assembly of FIG. 29;

FIG. 31 is a top plan view of the assembled assembly of FIG. 29, with portions removed, illustrating a second condition of the compression spring;

FIG. 32 is a side plan view, with portions removed, of the assembled assembly of FIG. 31;

FIG. 33A is a top plan view of the assembled assembly of FIG. 29, with portions removed, illustrating a nearly empty condition of the cartridge reservoir and the compression spring;

FIG. 33B is a side plan view, with portions removed, of the assembled assembly of FIG. 33A;

FIG. 34A is a top plan view of the assembled assembly of FIG. 22, with portions removed, of a further embodiment of a compression spring that assists throughout fluid delivery from a cartridge reservoir;

FIG. 34B is a side plan view, with portions removed, of the assembled assembly of FIG. 34A;

FIG. 35 is an exploded view, in perspective, of the pump unit and cartridge reservoir of the assembly of FIG. 22 just prior to the loading of the cartridge reservoir into the pump unit;

FIG. 36 is a perspective view of the pump unit and cartridge reservoir during the loading of the cartridge reservoir into the pump unit;

FIG. 37 is a perspective view of the pump unit and cartridge reservoir after the loading of the cartridge reservoir into the pump unit;

FIG. 38 is a perspective view of the pump unit and base of the assembly of FIG. 22 during the placement of the pump unit onto the base;

FIG. 39 is a bottom plan view of the assembled assembly of FIG. 22 shown during a priming process;

FIG. 40A is an exploded side plan view of the infusion device and an inserter for deploying the device in accordance with further aspects of the present invention;

FIG. 40B is an exploded view, in perspective, of the infusion device and inserter of FIG. 40A;

FIG. 41A is a perspective view of the infusion device and inserter of FIG. 40A after the infusion device has been loaded into the inserter;

FIG. 41B is a perspective view of the infusion device and inserter of FIG. 40A after the infusion device has been loaded into the inserter and after a protective cannula cover and one adhesive cover have been removed from the device;

FIG. 42 is a side plan view of the inserter, with the infusion device therein, against a patient's skin ready to deploy the device on the patient;

FIG. 43 is a side plan view of the deployed device having an insertion needle removed therefrom;

FIG. 44 is a perspective view of the insertion needle being safely stored in a cannula protector of the assembly of FIG. 22 for sharps disposal;

FIG. 45 is a perspective view of the deployed device on a patient's skin;

FIG. 46 is a perspective viewing which may be interpreted as showing either a pump unit being removed from a deployed base or a replacement pump unit being placed on a deployed base; and

FIG. 47 is a perspective view which may be interpreted as showing either a replacement pump being placed on a replacement base or a partially used pump unit being placed on a replacement base prior to deployment of the replacement base.

### DETAILED DESCRIPTION OF THE INVENTION

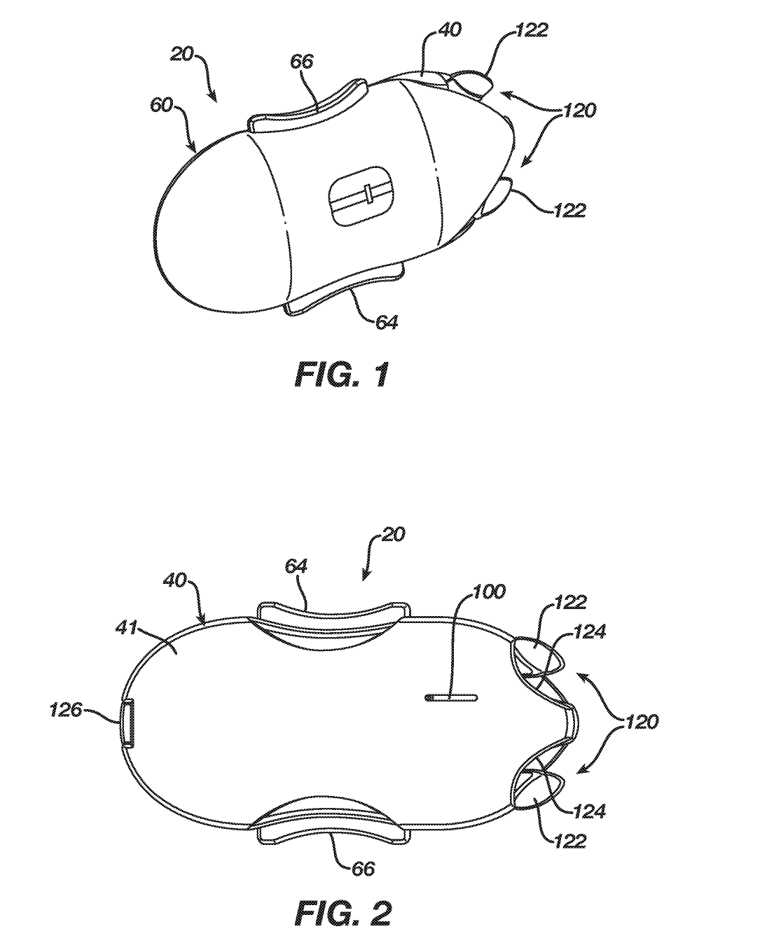

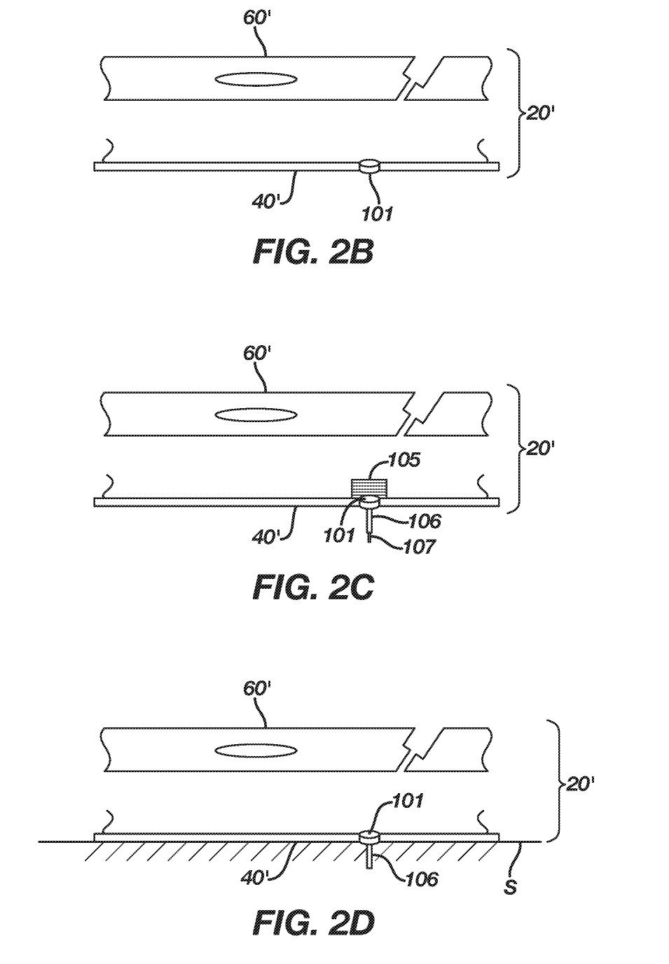

Referring now to FIGS. 1 and 2, they show a drug infusion system 20 according to a first embodiment of the invention. The system 20 generally includes a lower base portion 40 and a reusable drug dispenser portion 60. As will be seen subsequently, the reusable portion is arranged to be releasably attached to the base portion 40. In FIGS. 1 and 2, the base portion 40 and reusable portion 60 are fully engaged or joined together.

The base portion 40 includes a base surface 41 which preferably is coated with an adhesive for attaching the base portion 40 to the skin of a wearer in need of the drug, such as insulin, to be delivered by the system 20. To that end, the base 40, in a manner to be fully described herein after, is arranged to receive a cannula 100 which, when deployed, extends from the base surface 41 to beneath the skin of the wearer for subcutaneous delivery of the drug. The reusable dispenser portion includes a reservoir (not shown) for containing the drug and a pump (not shown) that, when actuated, pumps the drug from the reservoir to the cannula for delivery. As will be seen subsequently, when the base 40 and reusable portion 60 are joined together, a coupling arrangement provides an antiseptic connection there between. Also, the cannula 100 is a part of a cannula set which may be replaced in the base 40 when the reusable portion 60 is removed.

To actuate the pump, the reusable portion 60 includes a pair of actuator buttons 64 and 66. Preferably, the actuator buttons are arranged with the pump and other operative internal components of the reusable portion 60 so that concurrent depression of the actuator buttons 64 and 66 is required to actuate the pump. Infusion devices having such functionality are fully described, for example, in copending application Ser. No. 12/147,314 filed Jun. 26, 2008 for DISPOSABLE INFUSION DEVICE WITH PRIME INDICATOR, which application is assigned to the assignee of the present invention and incorporated herein by reference in its entirety. The pump for this or any of the subsequent embodiments may be any one of acceptable drug delivery pumps which may include, for example, a piston pump, a peristaltic pump, a screw pump, a membrane pump, a metering device, and a gas driven positive displacement pump.

The base 40 and reusable portion 60 are releasably fixed together by a latch assembly 120. In accordance with this embodiment, the latch assembly 120 includes a male part including projections 122 carried by the reusable portion 60 that are snappingly received within slots 124 of the base 40. A further snap-action latch 126 is provided at the distal end of the system 20 to complete the confinement of the reusable portion 60 on the base 40.

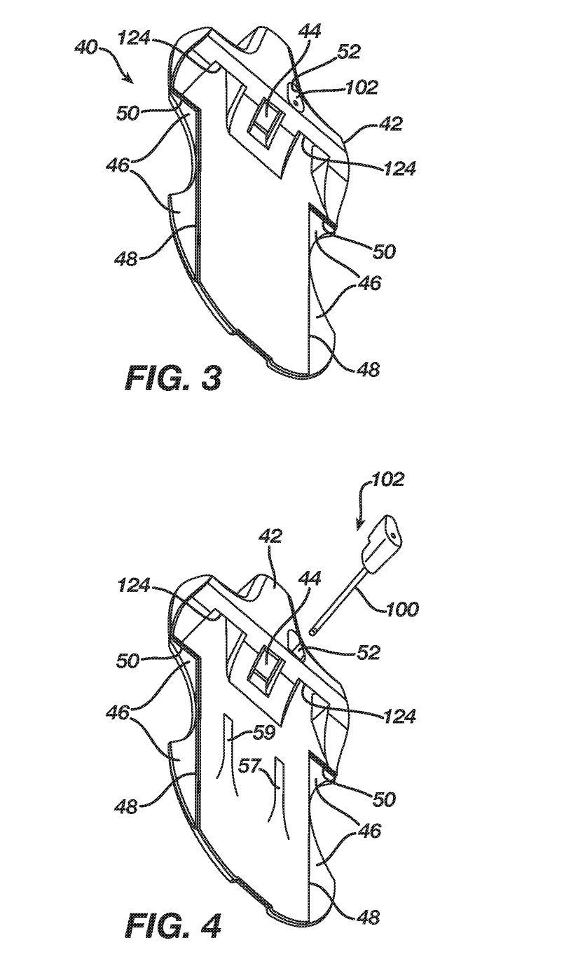

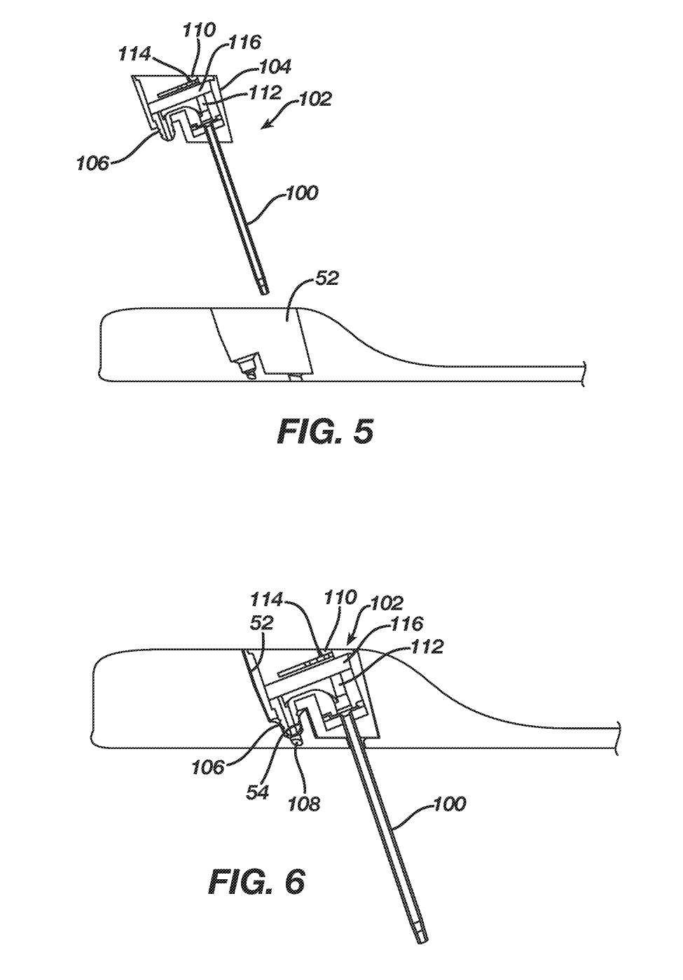

Alternatively, as shown in FIG. 2B, the system 20′ comprises a reusable drug dispenser 60′ and a base portion 40′, which is releasably attached to the combined pump unit and reservoir 60′. Similar to the other embodiments in this application, the base 40′ comprises an adhesive layer (not shown) configured to adhere to the skin of a wearer. The base also comprises a cannula well (similar to element 52 in FIGS. 3 and 4) disposed in the base. Alternatively, the cannula well may be disposed in the reusable drug dispenser 60′. To that end, the cannula set 102 (shown in FIGS. 5 and 6) is used to drive cannula 100 through cannula exit port 101.

FIG. 2C shows, in accordance with this embodiment, a cannula 106 that is provided as part of the base 40′. In this embodiment, the base comprises a needle handle 105 covering the cannula port 101 on proximal (non-skin) side of the base. Needle handle 105 is attached a to detachable drive needle 107, which is located on the distal side of the base 40′. The drive needle 107 is in turn held within cannula 106 which is affixed to the distal side of base 40′. Drive needle 107 is configured to introduce the cannula 106 into the skin.

In use, the wearer pushes the base 40′ against the wearer's skin, such that the needle 107 penetrates the skin. The cannula 106 is carried by the needle 107 through the tissue to beneath the skin. During this process and substantially simultaneously, the adhesive layer of the base 40′ will make contact with and adhere to the skin. FIG. 2D illustrates the assembly after the detachable drive needle is removed. This leaves the base 40′ attached to the skin S, and the cannula 106 extending through tissue beneath the skin.

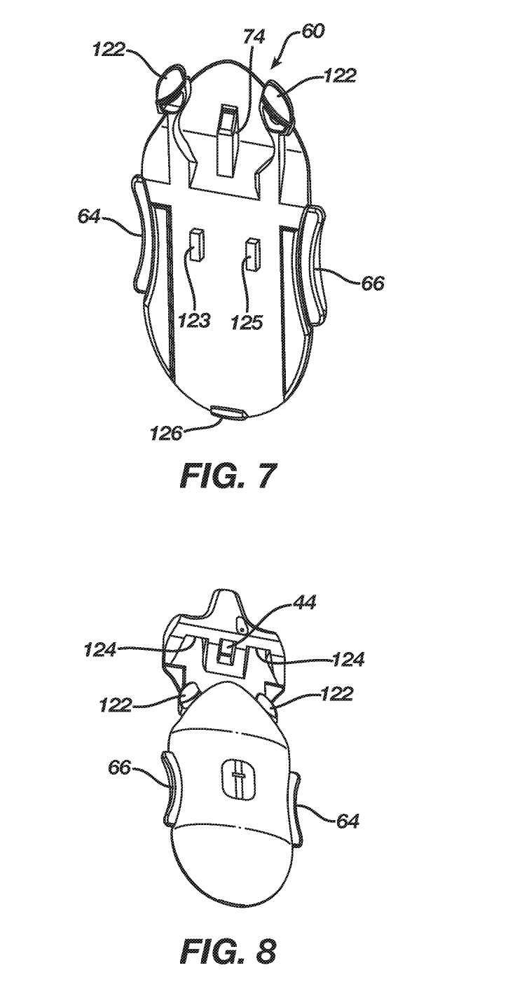

The perspective views of FIG. 3 show the base 40 in greater detail. Here it may be seen that the base 40 includes a head portion 42. The head portion 42 includes the slots 124 that snappingly receive the projections 122 (as shown in FIGS. 7 and 8) of the reusable portion 60 when the base 40 and reusable portion 60 are joined together. The head portion also includes an opening 44 into which a coupling part of the reusable portion 60 is received to establish the antiseptic connection between the base 40 and the reusable portion 60.

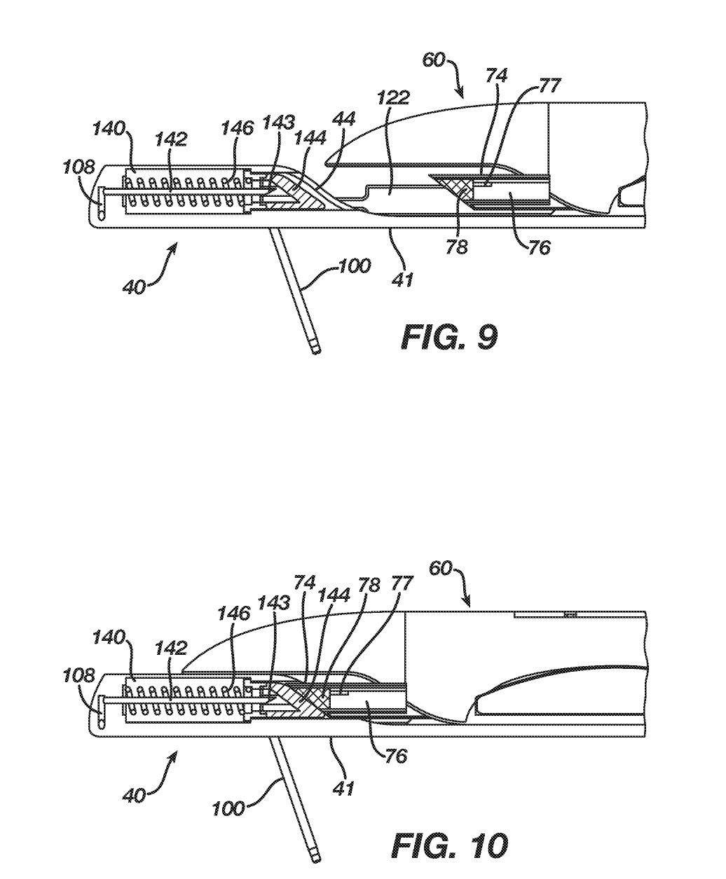

The base 40 further includes relieved surfaces 46 that form resulting shoulders 48 and 50. The shoulders 48 and surfaces 46 form guides to guide the reusable portion 60 into proper alignment with the base 40 as they are joined together. The shoulders 50 provide a stop which is engaged when the base 40 and reusable portion 60 are finally snapped together. The opening 44 may also be formed in its proximal portion as a channel that mates with coupling projection 74 as shown in FIGS. 7 and 9 to guide the aligned base and reusable portion into final and precise alignment for accurate attachment. Additionally or alternatively, grooves 57,59 in FIG. 4 may mate with projections 123,125 shown in FIG. 7 to help guide the two segments together in proper alignment.

As best seen in FIG. 4, the base 40 further includes a well 52 that is arranged to receive a cannula set 102 that includes the cannula 100. When the cannula set 102 is deployed, the cannula 100 is resultingly connected to an inlet within the head portion 42 and accessible through the opening 44 by the reusable portion.

The cannula set 102 and details of its deployment will now be described with particular reference to FIGS. 5 and 6. The cannula set 102 generally includes the cannula 100 and a cannula carrier 104. The cannula carrier is dimensioned to fit accurately in the cannula well 52 of the head portion 42 of the base 40 (FIG. 4). The carrier 104 includes a receiving pike 106 which is received by a correspondingly shaped feature 54 of the well 52. The feature 54 is in fluid communication with a conduit 108 through which the drug, such as insulin, is caused to flow by the pump. The drug hence flows through the conduit 108, through the feature pike 106, and to the cannula 100 for delivery.

The carrier further includes a port 110 through which a deployment needle (not shown) may be inserted. Prior to cannula deployment, the deployment needle extends through the port 110, through a passage 112, and through the cannula 100. The use of a deployment needle to subcutaneously place a cannula is described in greater detail in application Ser. No. 12/147,295 titled DISPOSABLE INFUSION DEVICE WITH AUTOMATICALLY RELEASABLE CANNULA DRIVER concurrently owned by applicant and incorporated herein in its entirety. The cannula set 102 is thus carried on the deployment needle. When the cannula set is deployed, the needle is retracted leaving the cannula set deployed as shown in FIG. 6.

To preclude direct access to the cannula 100 through the port 110 after deployment needle removal, the cannula set further includes a port cover 114. The port cover is preferably formed of resilient material and is arranged to spring over and block the port responsive to the drive needle being withdrawn from the port. Such a port cover is fully described, for example in co-pending application Ser. No. 12/147,306 filed Jun. 26, 2008 for DISPOSABLE INFUSION DEVICE WITH CANNULA PORT COVER, which application is assigned to the assignee of the present invention and incorporated herein by reference in its entirety. The port cover 114 together with a top 116 of the carrier 104 form a top sealing member of the carrier 104.

FIG. 7 shows the bottom view of the reusable portion 60. Here it may be seen that the reusable portion 60 includes a coupling projection 74 that is arranged to be received by the opening 44 of the base 40 when the base 40 and reusable portion are attached. It may also be seen that the reusable portion 60 includes the latch 126 at its distal end to complete confinement of the reusable portion 60 on the base 40.

FIG. 8 shows the reusable portion 60 being attached to the base 40. The projections 122 are aligned with and ready to be captured by the slots 124. When the reusable portion 60 reaches its final position on the base 40, it will cover essentially all of the base including the head portion 42 (FIGS. 3 and 4) as shown in FIG. 1.

FIGS. 9-11 show the establishment of the fluid coupling between the base 40 and the reusable portion 60 as the reusable portion is brought into engagement with the base. FIG. 9 shows the base 40 and reusable portion 60 prior to engagement.

Here the base 40 may be seen to include an inlet chamber 140. Extending through the inlet chamber 140 is a needle 142 that forms an inlet to the base 40. The needle 142 has a sharpened distal tip 143. The distal end of the inlet chamber 140 is sealed with a self sealing, penetrable, barrier or septum 144. A spring 146 urges the septum 144 in the distal direction. The reusable portion 60, in turn, includes a conduit 76 within the coupling 74. The coupling is sealed with a self sealing, penetrable, barrier or septum 78. Immediately adjacent the septum 78 is a one-way valve 77 to vent the conduit 76. This permits the drug, such as insulin, to be primed within the conduit so as to be in contact with the septum to eliminate air bubbles which might otherwise form.

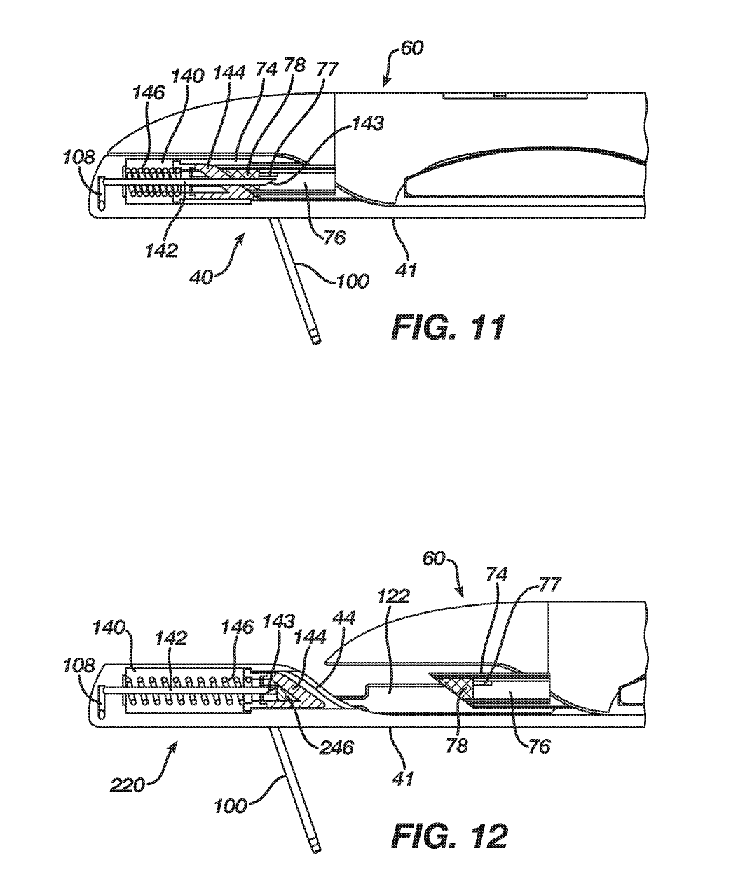

FIG. 10 shows the base 40 and reusable portion 60 just as they engage. Here, it can be seen that the coupling 74 has entered the opening 44 and that the barriers 144 and 78 have engaged each other. When the reusable portion 60 reaches its final position on the base 40 as shown in FIG. 11, the tip 143 of the inlet needle 142 has pierced through the septum 144 and the septum 78 to enter the conduit 76. The spring 146 is also compressed. As a result, a sealed fluid connection is established from the conduit 76 in fluid communication with the pump, through the inlet needle 142, through the conduit 108, and to the cannula 100 for drug delivery.

When it is necessary to remove the reusable portion 60 from the base 40, as the reusable portion 60 is pulled from the base 40, the compressed spring 146 forces the septum 144 distally until it once again seals the inlet chamber 140 as shown in FIG. 9. In addition to the inlet of the base 40 being sealed, the needle 142 is safely retracted back into the inlet chamber 140 to protect the wearer from being accidently pierced by the needle 146. More specifically, the opening 44 to the inlet chamber 140 may be made small enough to eliminate the danger of even the smallest of fingers of children, for example, from gaining access to the inlet chamber 140 and pushing the septum 78 in toward and being pierced by the needle tip 143 during the handling of the base 140.

FIG. 12 shows another drug infusion system 220 according to another embodiment of the invention. The system 220 is essentially identical to the system 40, previously described, and hence reference numerals for identical elements have been repeated herein and the description thereof is incorporated herein by reference. In addition to all of the elements of the system 40, the system 220 of FIG. 12 also includes an antiseptic wiper 246 within the inlet chamber 140 between the needle tip 143 and the septum 144. The antiseptic wiper 246 may be compressible foam or cotton or the like. It is provided for wiping the needle 142 whenever it is caused to pierce the septum 144 or be withdrawn through the septum 144 as when the base 40 and reusable dispenser 60 are joined or separated. The wiper is preferably formed of a substance that will not plug or clog the needle and that will not constitute an irritant to the wearer should trace amounts thereof be injected with the delivered drug.

An alternative (not shown) to the small wiper 246 illustrated would be a larger block of compressible foam or cotton impregnated with antiseptic solution, the cotton or foam contained with the bore 140 and located so that it would extend slightly back from the tip of the needle 142 to pipe most of the needle except the tip with antiseptic solution whenever the septum 144 is forced out beyond the tip of the needle 142 by spring 146.

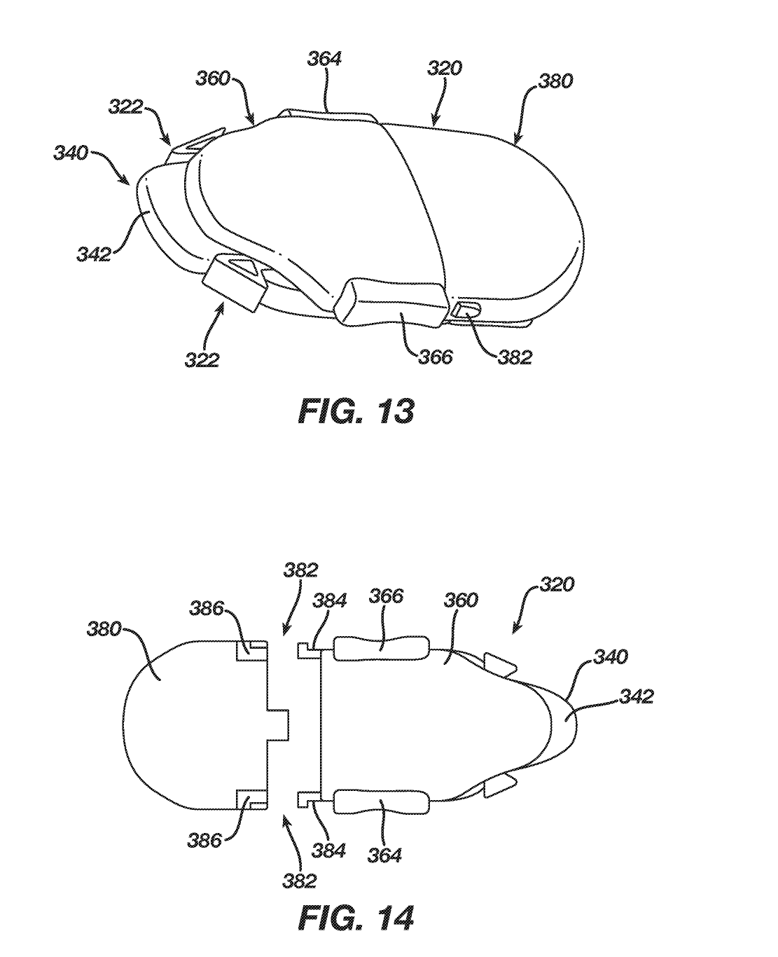

FIG. 13 shows still further embodiment of the present invention. Here, the drug infusion system 320 includes three primary components or portions; a base 340, a reusable pump unit 360, and a replaceable reservoir unit 380.

The base 340 may be similar to the base 40, previously described. To that end, it may also be arranged to receive a cannula set in its head portion 342 to establish fluid communication with the pump of the pump unit 360 in a manner as previously described.

The pump unit 360 is maintained on the base 340 by way of snap action latches 322 of the type previously described. The pump unit 360 includes actuator buttons 364 and 366 which, as previously described with respect to previous embodiments, are preferably arranged to cause drug delivery upon the concurrent depression of the actuator buttons 364 and 366.

The reservoir unit 380 is maintained on the base 340 by way of side snap action latches 382. The reservoir unit 380 is preferably prefilled prior to deployment in the system 320. More particularly, the reservoir unit 380 may be provided as a stand alone item from a drug manufacturer under prescription and not require any special handling by the patient except for its deployment on the base 340 in engagement with the pump unit 360. Alternatively, the reservoir unit may also be deployed on the reusable drug dispenser portion.

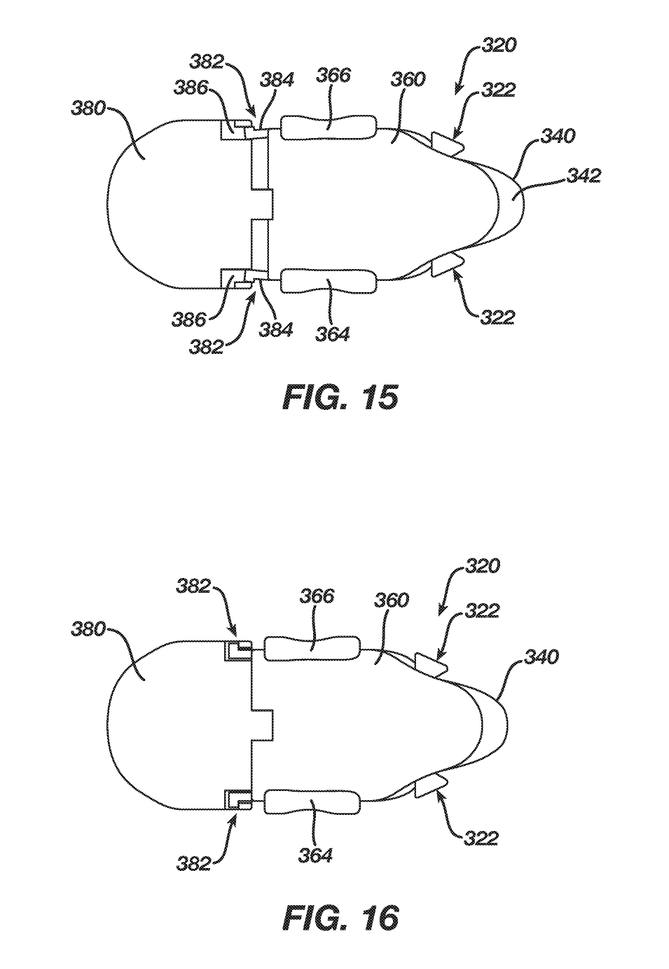

FIG. 14 shows the pump unit 360 and reservoir unit 380 in alignment for engagement. The side snap action latches 382 each comprises a latch projection 384 carried by the pump unit 360 and a receiving slot 386 formed in the reservoir unit 380. As may be seen in FIG. 15, to join the pump unit 360 with the reservoir unit 380 on the base 340, it is only necessary to advance the latch projections 384 into the receiving slots 386. Once this is accomplished, the system is fully engaged as shown in FIG. 16.

Antiseptic coupling of the base 340 and pump unit 360 and of the reservoir unit 380 and pump unit 360 may each be accomplished by employing dual septa and penetrating inlet needles as previously described. However, a vent need not be required for the antiseptic coupling of the reservoir unit 380 and the pump unit 360 because the reservoir unit may be manufactured to have the liquid drug, such as insulin, already immediately adjacent its sealing septum to prevent air bubble formation.

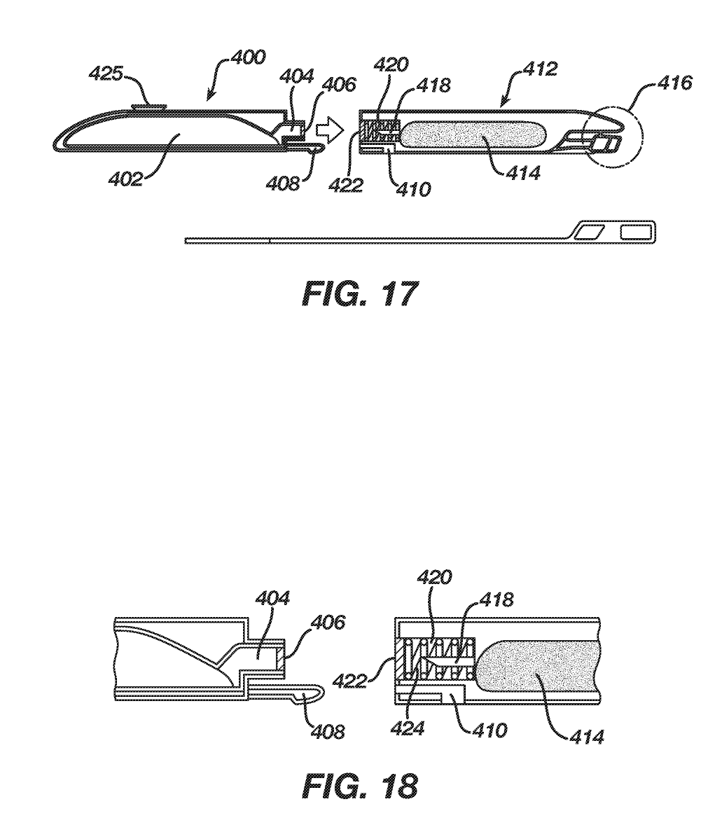

A more detailed description of the attachment mechanism of the reservoir portion and the pump portion may be seen in FIGS. 17 and 18. The reservoir portion 400 contains a collapsible reservoir 402 which is fluidly connected to an outlet bore 404. The collapsible reservoir may be characterized as a reservoir having a volume that decreases as fluid is expelled therefrom. Such a reservoir may be formed, for example, from flexible materials, or from rigid materials, having an internal moving component that decreases the volume within. The outlet bore is sealed with a piercable septum 406. The reservoir portion is further provided with a male snap projection 408 which is configured to releasably mate with a female receptacle 410 in the pump portion 412.

The pump portion 412 contains a pump 414 shown here in representative form. As stated earlier, any suitable pump may be employed. The pump portion contains on its distal end 416 all the mechanisms necessary to mate and form a detachable fluid connection with the base as described in detail above. In addition it contains a piercing needle 418 in an inlet bore 420. Located at the end of the bore is a sealing septum 422. A biasing mechanism, such as spring 424 urges the septum outward within the inlet bore 420. The septum is movable with the bore, and when the two septa 406,422 are urged against each other, the inlet bore septa slides back over the piercing needle 418 which simultaneously pierces the reservoir septum and forms a fluid connection between the reservoir and the pump.

As with the previous connection between the reusable portion and the base, an antiseptic member may be provided within the bore and surrounding the needle 418 to wipe the needle between connections. It is also to be noted that the previous description of the connection between the reservoir portion and the pump portion illustrated a side releasable snap configuration similar to the snap attachment between the reusable portion and the base, and in the embodiment shown in FIGS. 17 and 18, a bottom releasable attachment is illustrated. Likewise a top releasable attachment can easily be configured similar to the bottom releasable attachment shown.

The reservoir portion may be provided with a collapsible reservoir and prefilled by the manufacturer, in which case no priming mechanism is needed. If it is filled by the user soon before use, as is described in detail in the applications incorporated by reference herein, a simple mechanism of venting would be required. A vent comprised of a hydrophobic vent covered with a one way valve as described for the reusable unit and located on the outlet bore near the septum, in combination with a method of applying pressure to the reservoir such as a pressure button 425 would suffice.

In another embodiment, the reservoir may be a commercially available cartridge, such as an insulin cartridge. Such cartridges may be specially manufactured to fit the device, or may be of the type that is presently commercially available for syringe-pen injection units.

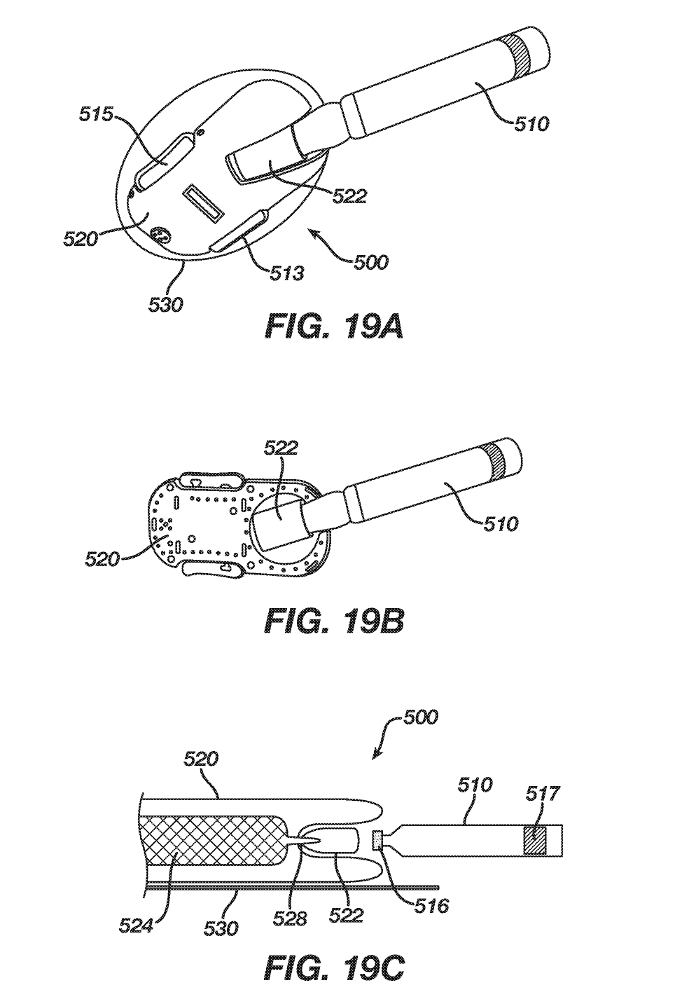

One possible embodiment using a commercially available cartridge (either pre-loaded or user loaded) is shown in FIGS. 19A through 19C. In this embodiment, as in the previous one, the pump unit of the reusable drug dispenser is configured to receive the reservoir. In accordance with this embodiment, the reservoir may be a commercially available cartridge, such as a glass syringe-pen cartridge (e.g., Humalog® or Humulin® sold by Lilly). As shown in FIGS. 19A and 19B, the device 500 comprises a cartridge reservoir 510, a reusable drug dispenser in the form of a pump unit 520 and an adhesive base layer 530. The cartridge reservoir 510 is received and maintained on the pump unit 520 by way of a receiving unit 522 that is in fluid communication with the cannula to the patient (not shown). As seen in FIG. 19C, the cartridge reservoir 510 typically contains a septum 516 at the distal end of the cartridge and a plunger 517 at the proximal end. The receiving unit 522 comprises a hollow penetrating inlet needle 528 configured to pierce the septum 516 of the cartridge. Receiving unit 522 is configured to extend beyond the tip of needle 528 such that the needle 528 is not exposed outside the device. This precludes a user (also defined as a wearer) from being accidentally pricked by the needle 528.

Needle 528 is in fluid communication with a pumping mechanism 524, which can be any of the pumping mechanisms previously described. The pumping mechanism (also called a pump) in turn is in fluid communication with a cannula (not shown).

In use, the user inserts reservoir 510 (if the pump unit is not already pre-loaded) into the receiving unit 522 with sufficient force to pierce the septum 516. Alternatively, the septum 516 may be pierced by the needle 528 by user activation after the user inserts it into the receiving unit. In some embodiments, it may be desirable that after inserting and securing a first reservoir, the pump unit is rendered unable to receive any subsequent reservoirs. This would make the reusable pump unit usable for the contents of just one reservoir. Once the needle 528 has pierced through the septum 516, the fluid contained within the reservoir is drawn via the needle 528, through the pumping mechanism 524 and out through the cannula (not shown) into the patient. The pumping mechanism 524 may be actuated by the concurrent depression of actuator buttons 513 and 515 (FIG. 19A) contained on the body of the device 500, as for example, on pump unit 520. When the user actuates the pumping mechanism 524, it draws fluid out of the reservoir 510, and delivers it into the cannula.

In this and subsequent embodiments, the pre-filled cartridge 510 may comprise a plunger element 517 as best seen in FIG. 19C. As the pumping mechanism 524 operates to draw liquid from the reservoir 510, the suction created serves to pull the plunger 517 towards the septum 516. The position of the plunger 517 may provide the user with a visual indication as to how much insulin remains within the reservoir. For example, some presently available insulin cartridges are equipped with visual volume indicators. Such markings may be calibrated to the amount of liquid left in the reservoir.

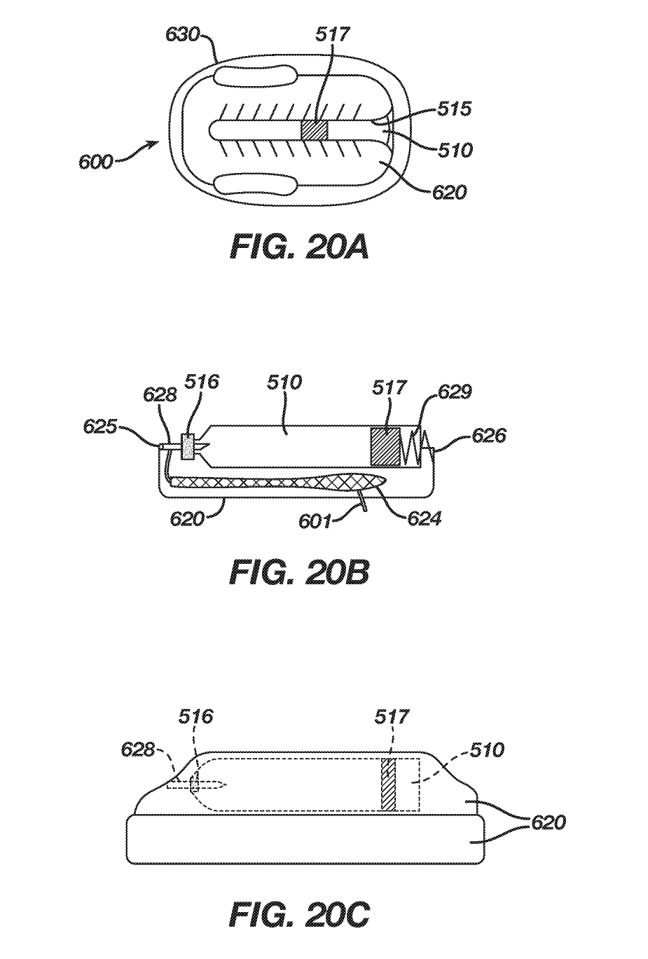

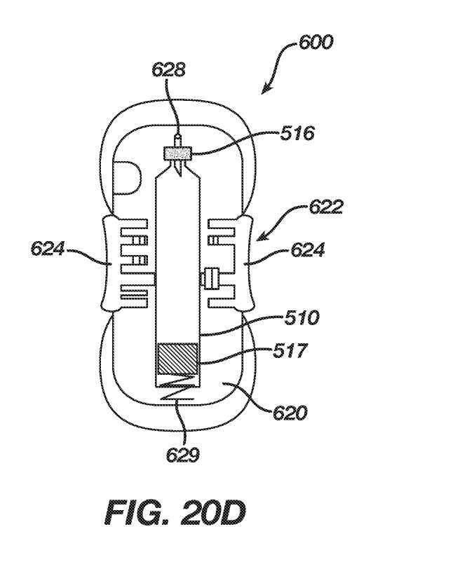

An alternative embodiment wherein a currently commercially available cartridge is employed as a reservoir is shown in FIGS. 20A through 20C. In this embodiment, a receiving unit is oriented to be in communication with a pre-filled cartridge that sits on top of the drug delivery device. Further, in this embodiment, the top of the device is configured to receive the pre-filled cartridge, for example, through a cavity that corresponds to the shape of the pre-filled cartridge.

As shown in FIG. 20A, the drug delivery device 600 comprises a reservoir unit 510, a reusable drug dispenser in the form of a pump unit 620 and a base adhesive layer 630. The reservoir unit 510 may be a pre-filled cartridge. Pump unit 620 may comprise an elongated cavity that corresponds to the shape of the pre-filled cartridge reservoir 510. For example, if the cartridge reservoir has a cylindrical configuration, the cavity preferably has a corresponding tubular configuration. The cavity 515 is configured to receive the reservoir 510 such that the reservoir is oriented parallel to the device.

As previously described in connection with FIG. 19C and in accordance with this embodiment, the cartridge 510 has a septum 516 at a first end and a plunger 517 disposed at a second, opposite, end. As seen in FIG. 20B, the cavity of the pump unit comprises first receiving unit 625 configured to receive the first end of the cartridge 510 and a second receiving end 626 that is configured to receive the second end of the cartridge 510. The receiving end 626 may further comprise a spring 629 that is configured to stabilize the reservoir within the cavity and/or to push the plunger 517 towards the septum 516. In pushing the plunger towards the septum, the spring may provide additional driving force to compliment the suction offered by the pump to expel the liquid from the reservoir into the needle 628 or to help the created suction overcome an initial resistance against movement of the plunger.

The penetrating inlet needle 628 is disposed in the receiving unit 625. As in previous embodiments, needle 628 is configured to pierce the septum 516 of the reservoir 510. In this embodiment, receiving unit 625 might be just the needle 628 anchored into the rest of the device. Alternatively, it may comprise the needle 628 and a suitable covering for the needle; for example, a tube, a hood or other such suitable sheath, to ensure that a user does not come into contact with the needle. Optionally, the cavity may be covered to provide a tubular opening into which the pre-filled glass cartridge may be located. Such an embodiment is contemplated in FIG. 20C. In this embodiment, an optional window (not shown) may be provided for visual indication of the amount of fluid left in the device.

As seen in FIG. 20D, the pumping unit 620 includes a pumping mechanism 622 that includes a pair of actuating buttons 624. The actuating buttons 624 are disposed within the pump unit 620 to accommodate the cavity that will house the reservoir 510. More specifically, the pumping mechanism 622 (also referred to as a pump) may be configured such that one actuating button resides on one side of the cavity, and the other actuating button resides on the other side of the cavity. The actuating buttons 624 may carry attendant pump features distributed equally on opposite sides of the cavity.

In use, the cartridge reservoir 510 is placed into the cavity 515 of the pumping unit 620 such that the septum 516 contacts and is penetrated by the penetrating inlet needle 628. The penetrating inlet needle 628 is in fluid communication with the pumping mechanism 622, which can be any of the pumping mechanisms previously described. The pumping mechanism in turn is in fluid communication with cannula 601 (FIG. 20B). The pumping mechanism is user actuated by, for example, the concurrent depression of the actuator buttons 624 on the body of the device 600, for example on pump unit 620. When the user actuates the actuator, the pumping mechanism 624 draws fluid out of the reservoir 510, and delivers it to the cannula. In embodiments employing spring 629, the spring may facilitate the drawing of fluid by the pump either by creating a continuous pressure throughout the course of use, or by helping overcome friction during the first actuation.

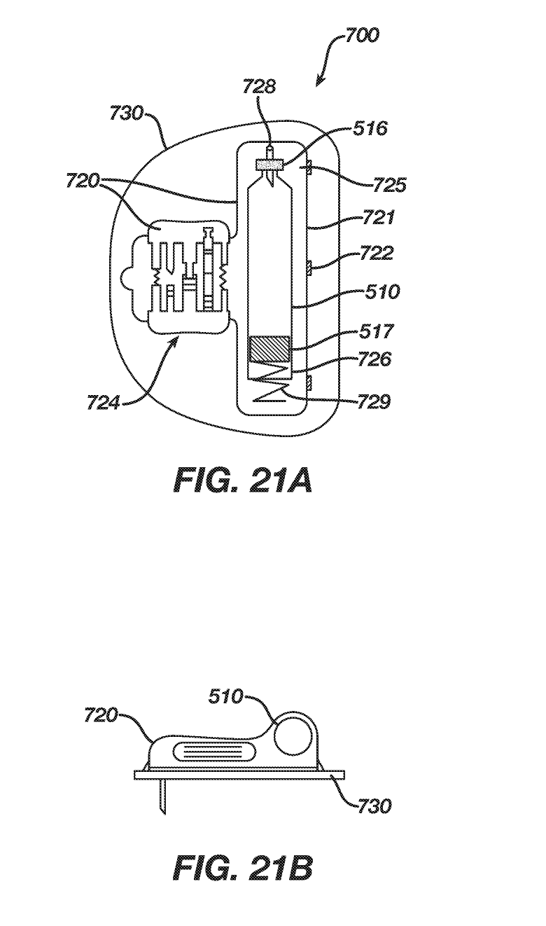

A further embodiment of the present invention is shown in FIGS. 21A and 21B. FIGS. 21A and 21B show an embodiment of the invention in top perspective and end perspective views, respectively. In this embodiment, a device 700 comprises a removable reservoir 510, a reusable drug dispenser in the form of a pump unit 720, and an adhesive base layer 730. Pump unit 720 comprises a pumping mechanism 724 (also referred to as a pump) and an additional encasing unit 721. Encasing unit 721 is configured to house the reservoir 510. The encasing unit 721 is oriented to one side of the pumping mechanism 724. This design lowers the vertical profile of the device by allowing the reservoir 510 to be housed beside the pumping mechanism 724. However, if profile is not a concern, the encasing unit 721 may be placed in any orientation relative to the device, as for example, on top of the device, as seen for example in FIG. 20C, where the reservoir is encased on top of the pump unit. The encasing unit 721 additionally comprises one or more securing mechanisms 722 which may take the form of locking tabs to secure the reservoir 510 within encasing unit 721. Optionally, a window (not shown) may be provided on the encasing unit 721 to allow visualization of the plunger position and hence the amount of fluid left in the cartridge.

As in the previous embodiments, the pump unit comprises a receiving end 725 and a base-end 726. The receiving end 725 comprises a penetrating inlet needle 728, which is configured to penetrate the septum 516 of the reservoir. The base end 726 optionally comprises a spring 729 that is configured to push the plunger 517 of the device, thereby assisting with fluid entry into the needle 728.

As in the previous embodiments, in use, a user places the reservoir 510 into the pump unit 720 such that the septum 516 contacts and is pierced by the needle 728. The needle 728 is in fluid communication with the pumping mechanism 724 which draws fluid out of the reservoir 510 and into the cannula (not shown). In embodiments employing spring 729, the spring facilitates the drawing of fluid by the pump either by creating a continuous pressure throughout the course of use, or by helping overcome friction during the first actuation.

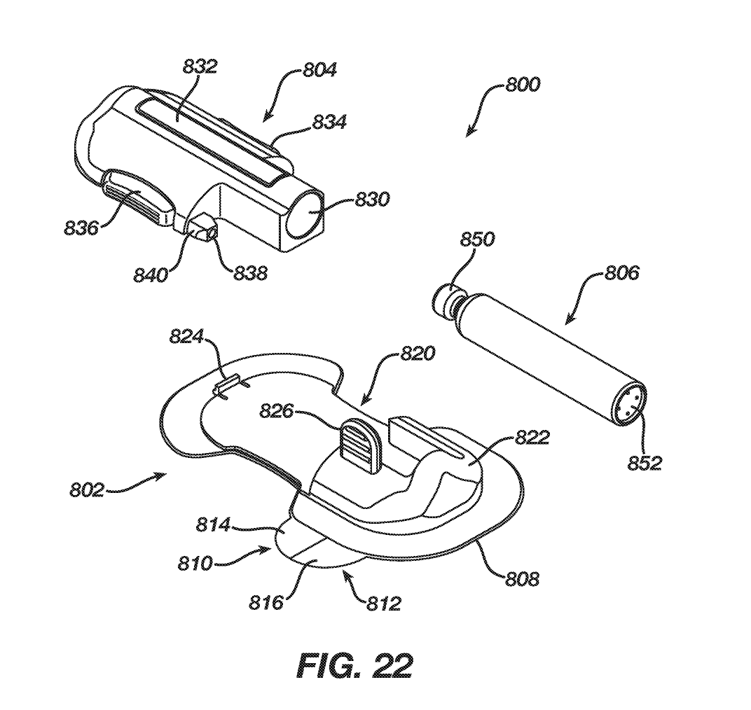

Referring now to FIG. 22, it shows another assembly 800 embodying the present invention in exploded and perspective view. As in prior embodiments, the assembly is a three component assembly including a base 802, a pump assembly 804, and a cartridge reservoir 806.

The base 802 includes a flexible web 808 which has an adhesive thereon to permit the base to be adhered to the skin of a patient. Covering the adhesive are two tabbed covers 810 and 812 including tabs 814 and 816 respectively. The tabs allow the covers to be readily peeled off to expose the adhesive just prior to deployment of the base against the patient's skin.

The base 802 further includes a receiving structure 820 secured to the top surface of the web 808. The receiving structure is arranged to detachably receive the pump unit 804 therein and includes a housing 822 arranged to receive and confine the forward end of the pump unit 804. The receiving structure further includes a latch 824 that releasably locks the pump unit 804 onto the base 802.

As will be seen subsequently, the base 808 includes a built-in cannula that extends from the adhesive side of the base 808. To facilitate deployment of the cannula as the assembly is adhered to the patient's skin, the base also includes an insertion needle of the type known in the art that extends through the cannula and carries it to a deployed position. As will also be seen subsequently, after the assembly is deployed, the insertion needle may be pulled out of the cannula and the housing. To that end, a handle 826 connected to the insertion needle is provided. After deployment of the assembly 800, the handled 826 may be grasped and pulled to remove the insertion needle.

The pump unit 804 includes an elongated cavity 830 for receiving the cartridge reservoir 806. The cavity, in accordance with this embodiment, has a tubular shape to correspond to the generally cylindrical shape of the cartridge reservoir 806 as may be noted in the drawing. The pump unit 804 may include a window 832, through which the amount of fluid left in the cartridge reservoir may be observed.

In accordance with prior embodiments, the pump unit 804 may contain any one of the pump mechanisms previously described herein. Actuation of the pump unit 804 may be achieved through a pair of actuating buttons 834 and 836. Preferably, the pump unit 804 is arranged so that concurrent depression of the actuating buttons 834 and 836 is required to actuate the pump unit 804.

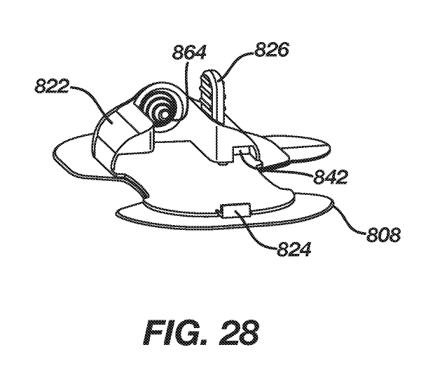

In accordance with this embodiment, when the pump unit 804 is actuated, a bolus of the fluid carried in the cartridge reservoir 806 is dispensed out of on outlet port 838 of the pump unit 804. The outlet 838 is defined by a fitting 840 that makes a fluid tight seal with a corresponding inlet 842 (FIG. 28) of the housing 822. The inlet 842 is in fluid communication with the cannula to cause the bolus of fluid to be delivered to the cannula.

The cartridge reservoir 806 may be of the type previously described. It includes a septum 850 at its distal end and a plunger 852 at its proximal end. The plunger, as in prior embodiments, is arranged to translate along the length of the cartridge reservoir as fluid is removed therefrom. The position of the plunger 852 may be seen through the window 832 to provide the wearer with an indication as to how much fluid is remaining in the cartridge reservoir 806.

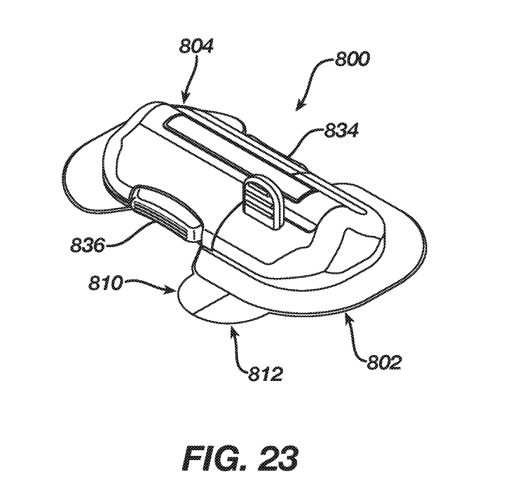

FIG. 23 shows the components of the assembly 800 assembled into an infusion device. The cartridge reservoir (not shown in FIG. 23) has been loaded into the pump unit 804. The pump unit 804 in turn has been releasably secured to the base 802. The assembly, after the tabbed covers 810 and 812 are removed, will be ready to be deployed on a patient.

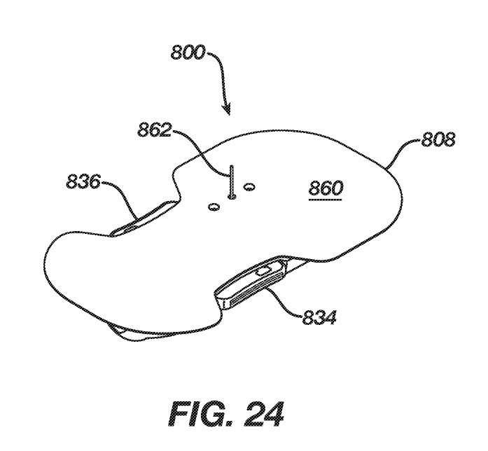

FIG. 24 shows the bottom of the device 800 after the tabbed covers are removed to expose the adhesive surface 860 of the flexible web 808. As may also be seen in FIG. 24, the cannula 862 extends through the flexible web 808. As will be seen herein after, the cannula 862 is protected by a protective cover that may be removed just prior to device deployment.

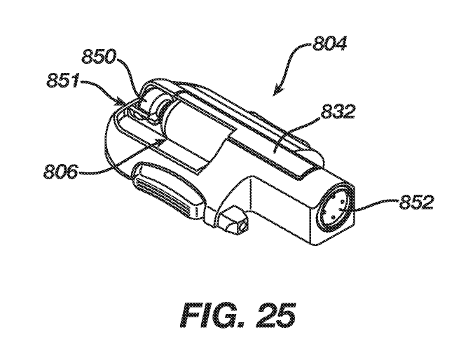

Referring now to FIG. 25, it is a perspective view, with portions cut away, illustrating a releasable lock of the cartridge reservoir 806 within the pump unit 804. Here the cartridge reservoir has been fully loaded into the pump assembly 804. A latch assembly 851 firmly holds the cartridge reservoir 806 in place while also permitting the cartridge reservoir 806 to be removed from the pump unit 804 if necessary or desired.

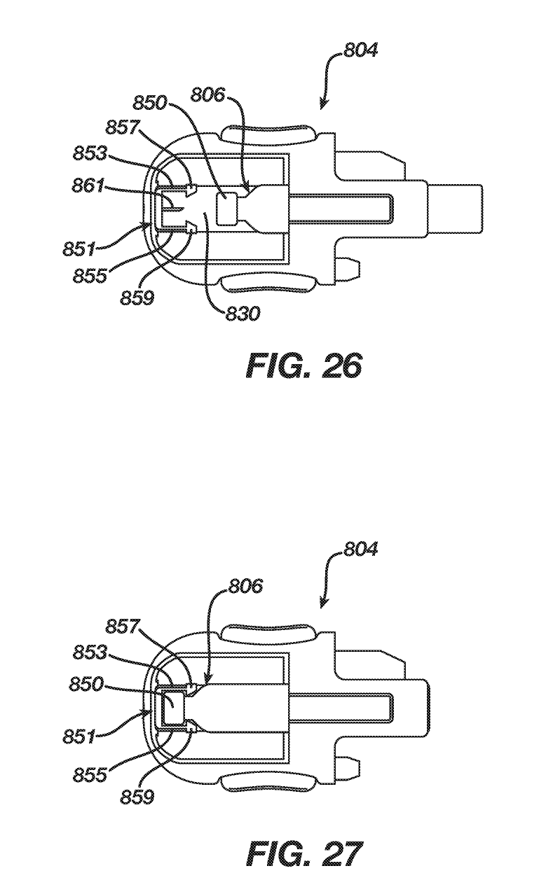

FIG. 26 illustrates the latch assembly 851 in greater detail. Here it may be seen that the latch assembly 851 is substantially U-shaped having extensions 853 and 855. The extensions 853 and 855 extend into the cavity 830 of the pump unit 804 and terminate in latching ends 857 and 859 respectively. The extensions 853 and 855 are of sufficient length to fully encompass the septum 850 of the cartridge reservoir 852 when the cartridge reservoir 852 is fully loaded into the pump unit 804.

FIG. 26 also shows a needle 861. The needle 861 serves to penetrate the septum 850 when the cartridge reservoir 852 is fully loaded into the pump unit 804. This provides the fluid connection between the cartridge reservoir 806 and the pump mechanism (not shown). Since the needle 861 is deep within the cavity 830 of the pump unit 804, protection against accidental contact with the needle is provided.

FIG. 27 shows the cartridge reservoir 806 after being loaded into the pump unit 804 and being releasably locked therein by the latch assembly 851. The septum 850 of the cartridge reservoir 806 is fully captured by the extensions 853 and 855 and their latching ends 857 and 859 respectively. The extension 853 and 855 are resilient for deflection to allow the septum 850 to enter past the latching ends 857 and 859. This also allows the septum 850 to be withdrawn past the latching ends 857 and 859 when removal of the cartridge reservoir 806 from the pump unit 804 is necessary or desired.

FIG. 28 is an end view, in perspective, of the base unit of the assembly of FIG. 22. Here, it may be seen that the housing 822 of the base 802 includes a spring 864 arranged to engage the plug 852 of the cartridge reservoir 806 (FIG. 22). Although a coiled spring is illustrated, it should be apparent to those skilled in the art that the spring may take different forms, such as for example, a leaf spring.

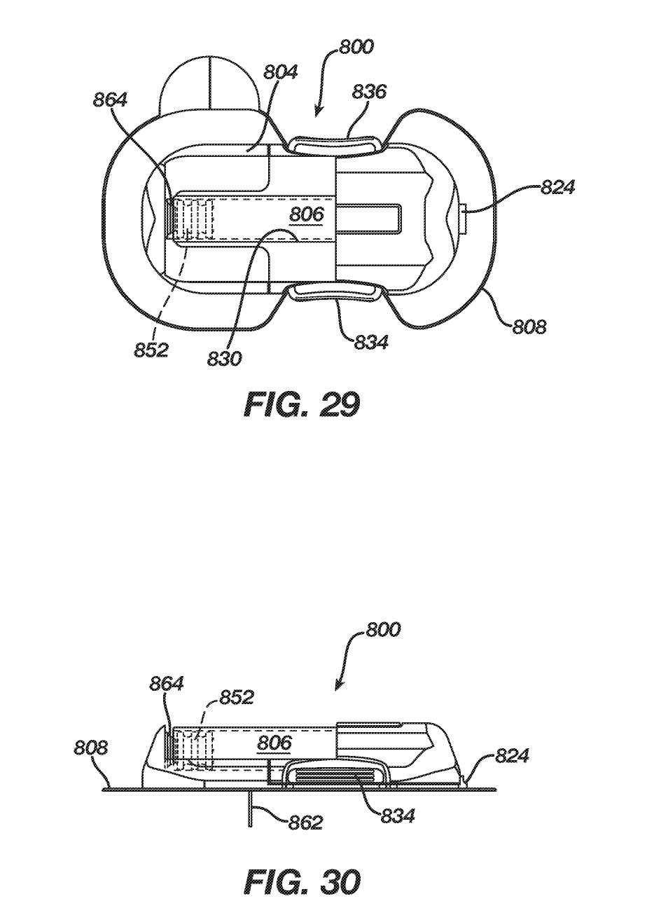

FIGS. 29-34 illustrate the functioning of the spring 864. As may be seen in FIGS. 29 and 30, when the cartridge reservoir 806 is originally received within the cavity 830 of the pump unit 804, the spring 864 contacts the plunger 852 of the cartridge reservoir 806. The spring 864 becomes compacted to store energy and is now ready to assist the pulling of fluid from the cartridge reservoir 806 during the first actuation of the device 800 to overcome any friction that may otherwise preclude movement of the plug 852 within the cartridge reservoir 806.

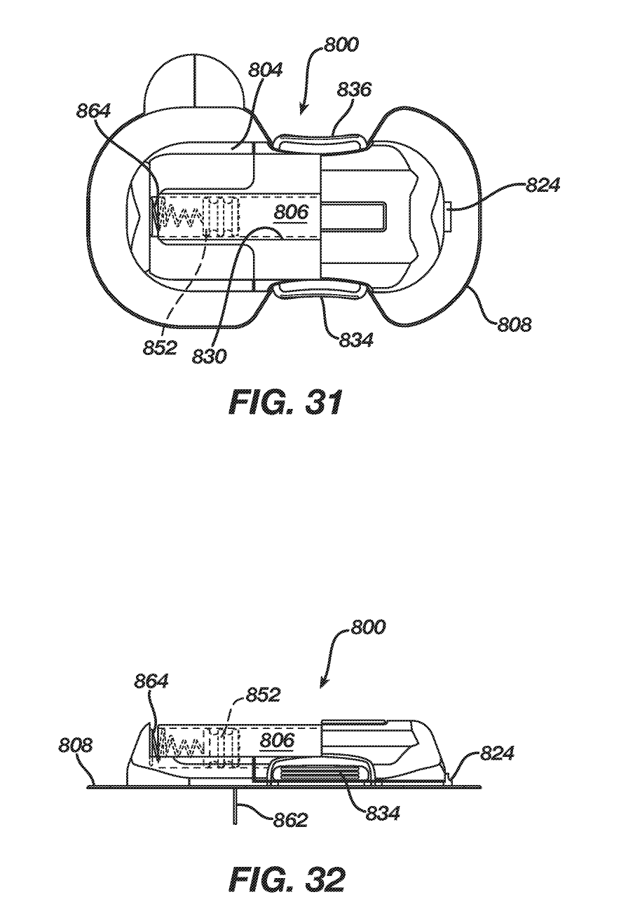

As may be seen in FIGS. 31 and 32, the spring 864 is of sufficient length so that as fluid continues to be drawn from the cartridge reservoir 806, it will assist in the movement of the plunger 852. In some embodiments, the spring may only be required to free the plunger 852 for its initial movement. In that event, further spring function may be unnecessary permitting the spring to have a shorter axial free state length.

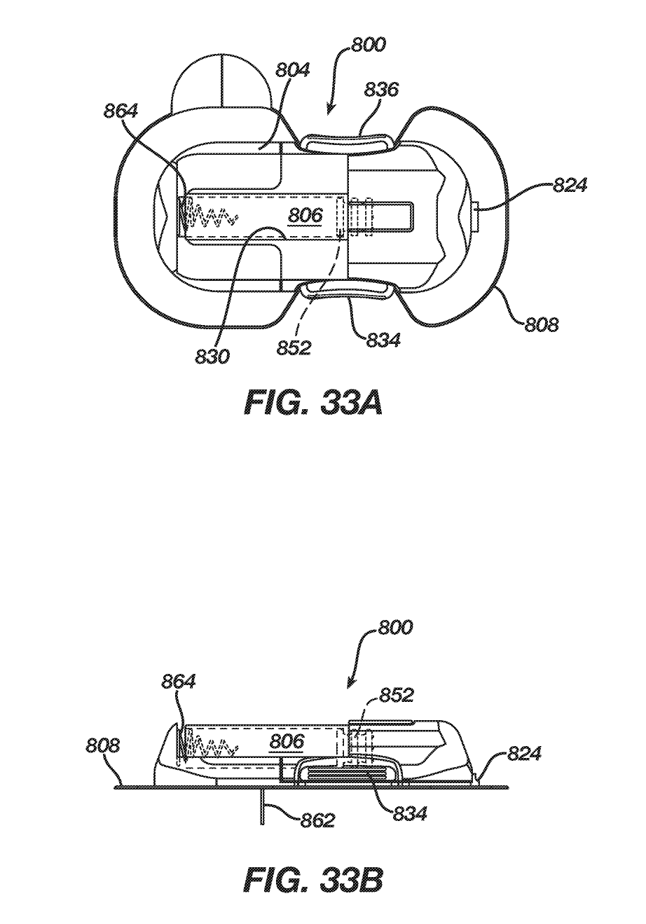

Eventually, as may be seen in FIGS. 33A and 33B, the cartridge reservoir 806 will be almost empty and the plunger 852 will have moved far enough within the cartridge reservoir that the spring 864 will project into the cartridge reservoir 806 and will have lost contact with the plunger 852.

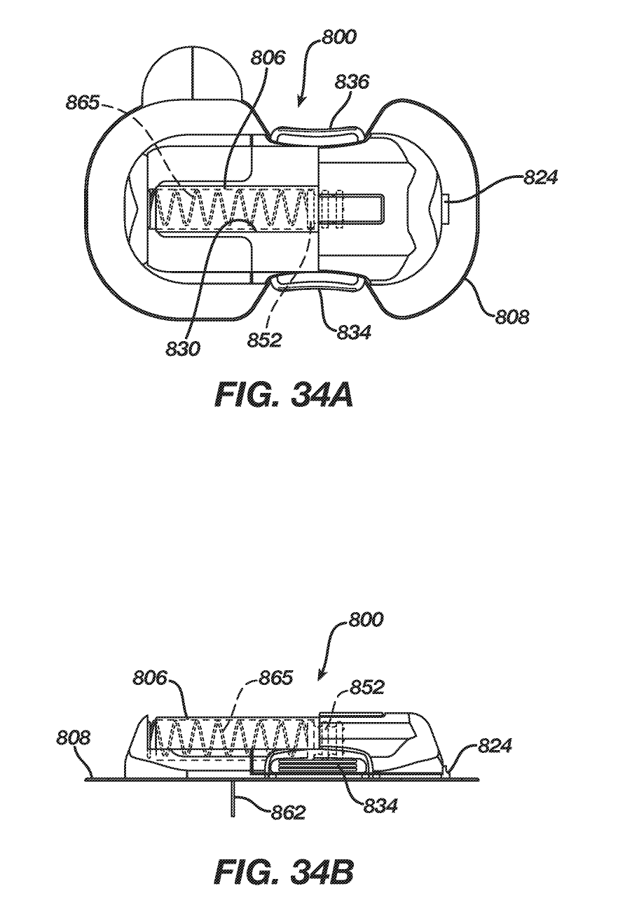

FIGS. 34A and 34B illustrate a further embodiment of the compression spring. Here, the compression spring 865 is of sufficient length to assist in the delivery of the fluid from the cartridge reservoir 806 until it is empty. As a result, the spring 865 remains in constant contact with the plunger 852 throughout its travel through the cartridge reservoir 806.

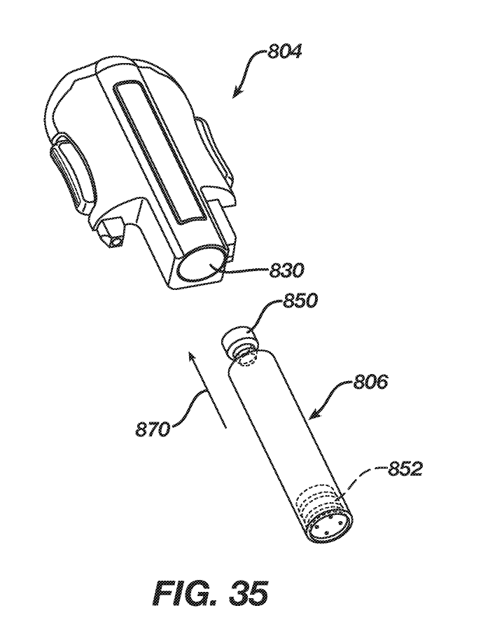

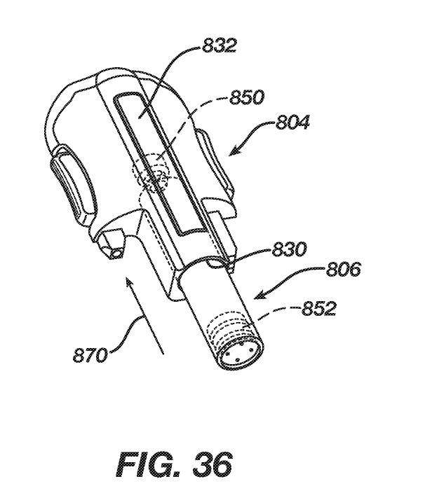

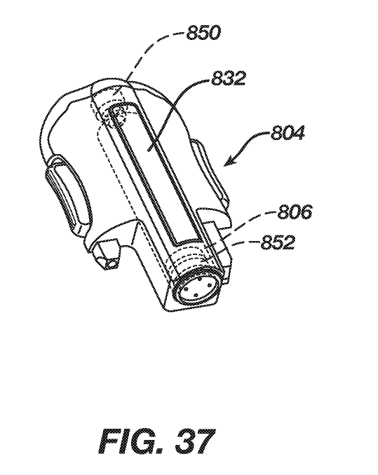

FIGS. 35-39 show the sequence of steps to be performed to make the assembly 800 of FIG. 22 ready for deployment. As may be seen in FIG. 35, the cartridge reservoir 806 is moved relative to the pump unit 804 in the direction of arrow 870 to insert the cartridge reservoir 806, septum 850 end first, into the cavity 830 of the pump assembly. As may be seen in FIG. 36, as the cartridge reservoir 806 is moved in the direction of arrow 870, the septum 850 end of the cartridge reservoir 806 may be viewed through the window 832 to monitor the cartridge reservoir 806 insertion process. When the cartridge reservoir 806 is fully inserted into the pump unit 804, the partial assemblage will appear as shown in FIG. 37.

Preferably, the pump unit 804 includes cartridge receiving structures of the type previously described herein including a septum piercing needle to pierce the septum 850 and connect the cartridge reservoir 806 to a pump mechanism. The plunger 852 of the cartridge reservoir 806 protrudes slightly from the proximal end of the pump unit 804 and is ready to contact a spring of the base as previously described.

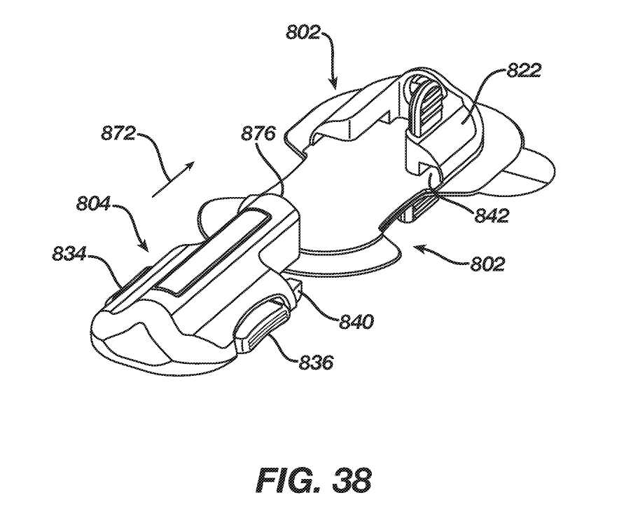

Next, the pump unit 804 is releasably joined with the base 802. As seen in FIG. 38, this is accomplished by sliding the pump unit 804 in the direction of arrow 872 until the proximal end 876 of the pump unit 804 is fully within the housing 822 of the base 802. As the proximal end 876 of the pump unit 804 enters the housing 822, the fitting 840 of the pump unit 804 will make a fluid tight seal with the inlet 842 of the housing 822 of the base 802. This having been accomplished, the assembled assembly 800 will appear as previously shown in FIG. 23.

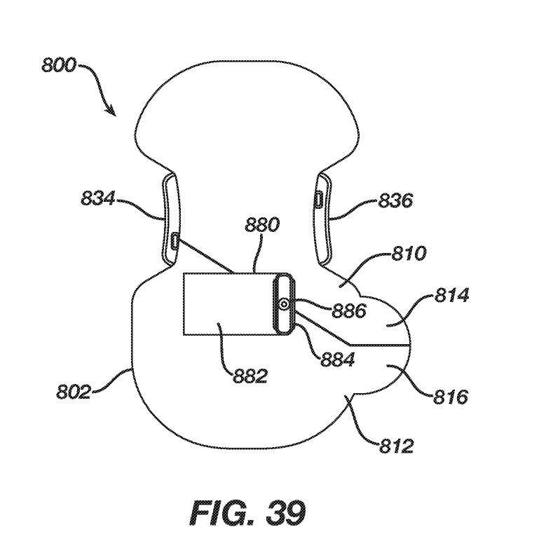

Now, priming of the fluid delivery system and removal of the adhesive covering tabbed covers 810 and 812 are required. This may be accomplished as shown in FIG. 39, which shows the bottom view of the device (i.e., the device 800 is turned over). A protective structure 880 protects the cannula from damage. More specifically, the protective structure 880 is substantially shaped and includes a horizontal portion 882 and a vertical portion 884 substantially transverse to the horizontal portion 882. The vertical portion 884 includes a bore 886 having the cannula therein.

To prime the fluid delivery system, the device 800 is actuated by depressing the actuator buttons 834 and 836 enough times to cause fluid to appear out of bore 886. When this occurs, it is known that the cannula and all of the fluid conduits from the cartridge reservoir to the distal end of the cannula are filled with fluid.

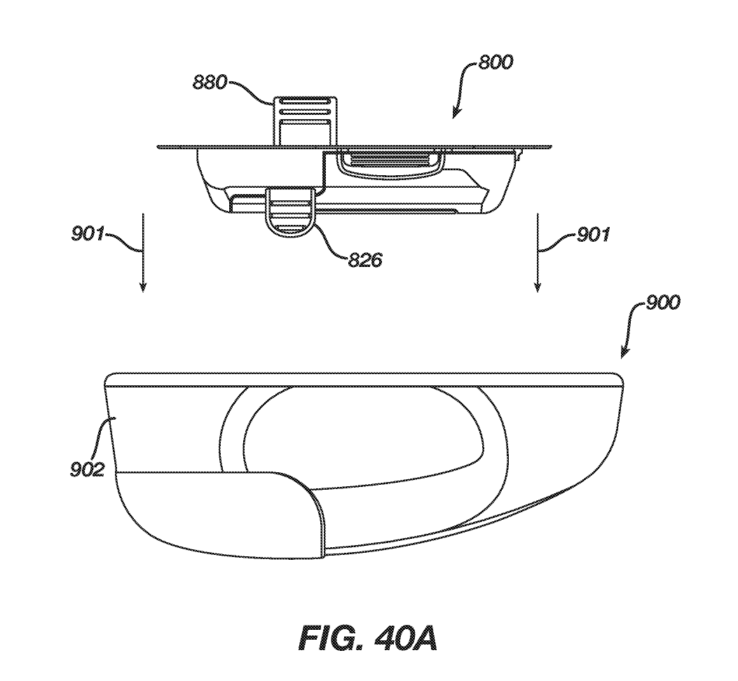

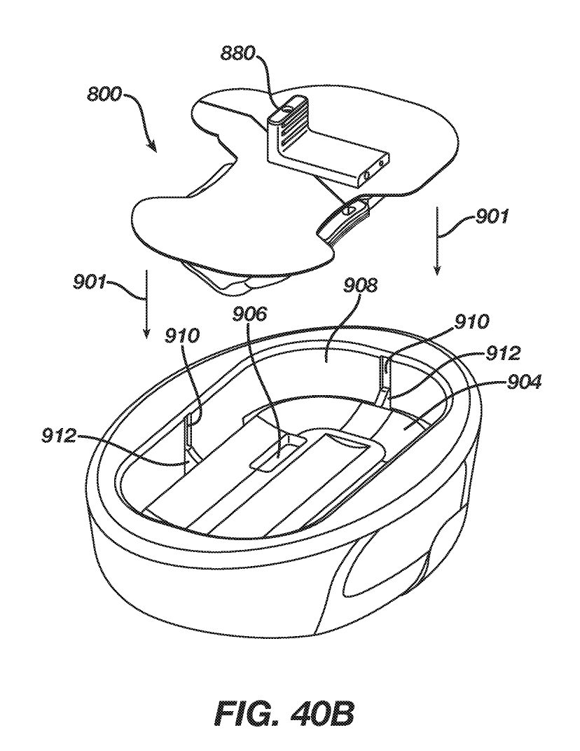

FIGS. 40A and 40B are exploded views of the infusion device 800 and an inserter 900 for deploying the device in accordance with further aspects of the present invention. The inserter 900 includes a housing 902 dimensioned to reach the device 800. The device 800 may thus be placed into the inserter 900 in the direction of arrows 901. The inserter housing 902 includes a moveable top 904 that has an inner surface contour that matches the general surface contour of the device 800. The top 904 has an opening 906 for receiving the insertion needle handle 826 that protrudes from the device 800. The inserter housing 902 has a side wall 908 that includes guide channels 910. The guide channels 910 slidingly receive guide extensions 912 that extend from the inserter top 904. The guide channels 910 and guide extensions 912 serve to controllably guide the translation of the top 904, and hence the device, during deployment of the device 800. To that end, the top may be manually driven by the user or the top may be driven by a mechanical drive force as may be provided by the stored energy of a drive spring, for example.

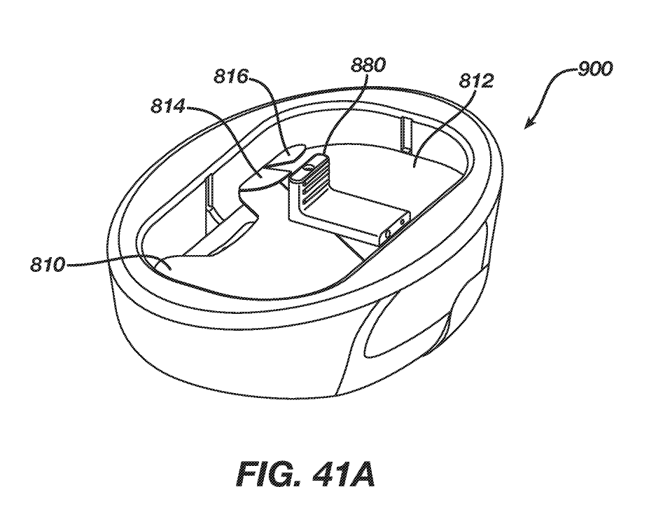

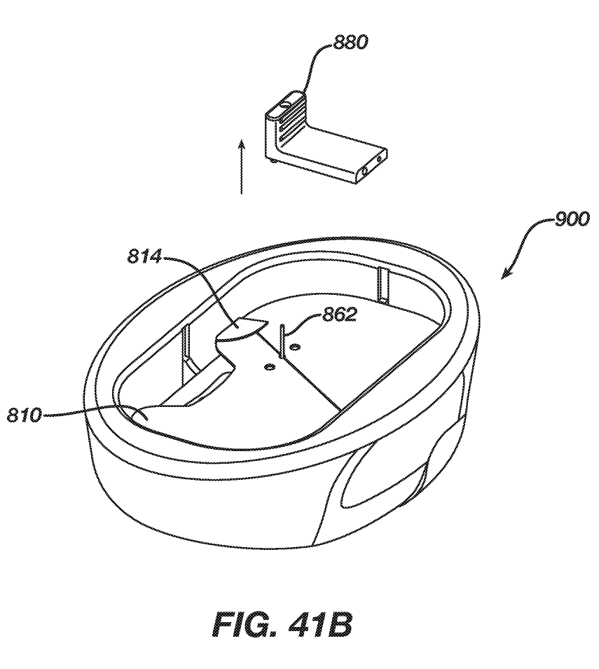

FIGS. 41A and 41B are perspective views of the infusion device 800 and inserter 900 after the infusion device has been loaded into the inserter. In the process of loading the device 800 into the inserter 900, the tabs 814 and 816 of the tabbed adhesive protective covers 810 and 812 respectively are turned-up for ready removal. FIG. 41B shows the cover 812 removed and cover 810 ready for removal. Also seen in FIG. 41B is the cannula protective structure 880 removed from the cannula. Once cover 810 is removed from the device 800, the device 800 will be ready for deployment with the inserter.

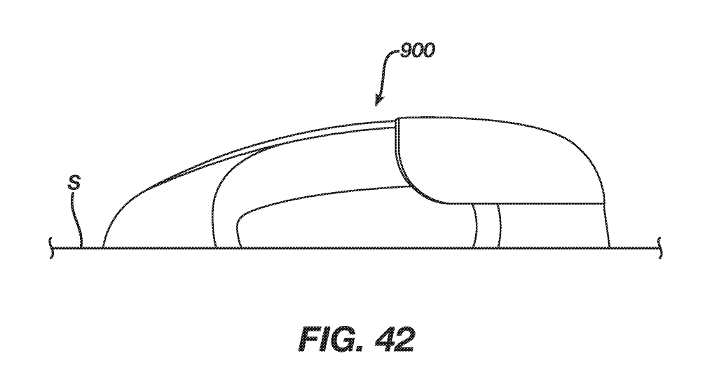

FIG. 42 shows that the inserter has been placed against the skin S of the patient. Now, upon actuation of the inserter, either by manual force or released stored force, the entire device will be driven to the skin of the patient. This will cause the cannula and insertion needle to penetrate the patient's skin and the adhesive surface of the base of the device to contact and be adhered to the patient's skin.

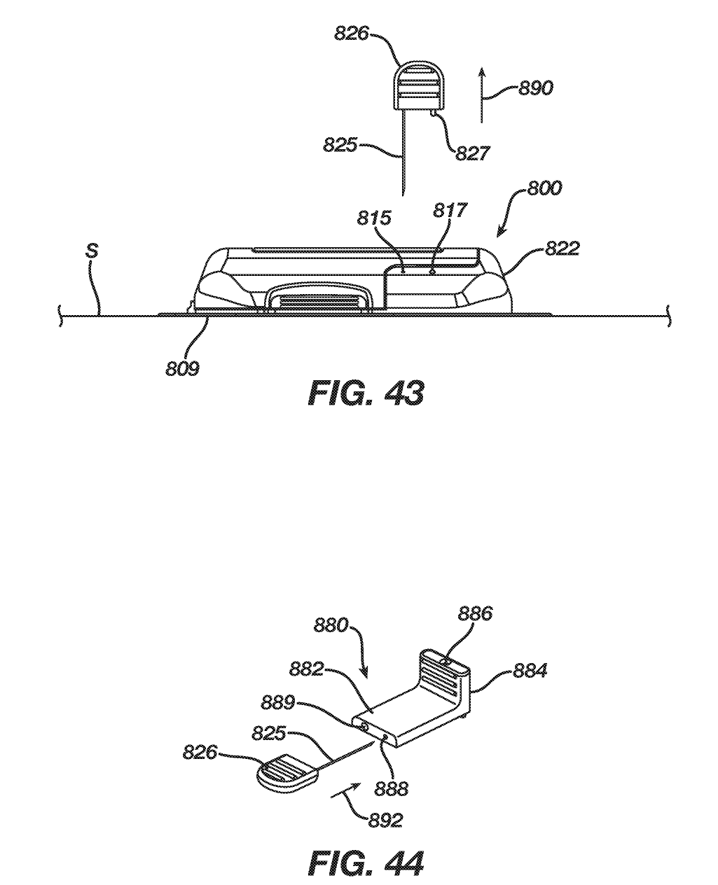

FIG. 43 shows the device 800 on the patient's skin S after the inserter has been removed. The base surface 809 of the device is adhered to the patient's skin. FIG. 43 also illustrates the insertion needle 825 being pulled from the device 800 and more specifically the cannula (not shown) through a hole 815 in the base housing 822 of the device 800 in the direction of arrow 890. The insertion needle is readily pulled by grasping the handle 826 of the insertion needle 825. As may be further seen in FIG. 43, the insertion needle handle 826 includes an alignment pin 827 that is being pulled from a corresponding hole 817 of the base housing 822.

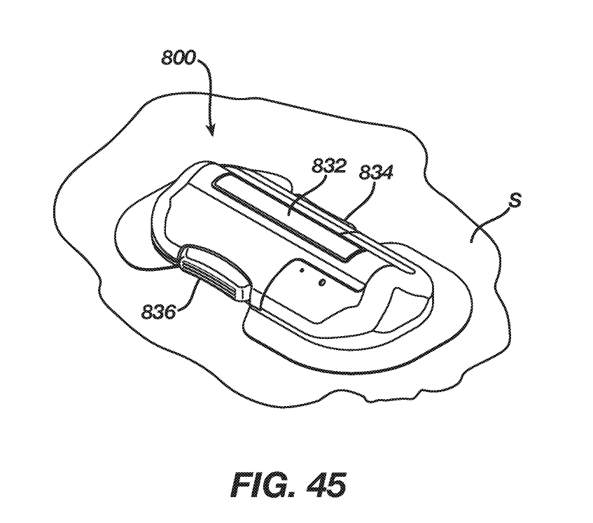

FIG. 44 illustrates a preferred manner of storing the insertion needle 825 once it has been removed from the device. Here it may be seen that the needle 825 is stored in the protective structure 880 previously used for protecting the cannula of the device. To that end, the protective structure includes a hole 888 within the horizontal portion 882 for receiving the needle 825 as the needle is inserted therein in the direction of arrow 892. The horizontal portion further includes a hole 889 for likewise receiving the alignment pin 827 (FIG. 43) of the insertion needle handle 826. The insertion needle is now ready for safe sharps disposal.

FIG. 45 shows the device 800 fully deployed on the patient's skin S and ready to provide a first bolus of liquid medicament to the patient. While the device is in use, the amount of medicament remaining in the cartridge reservoir may be discerned by simply looking through the window 832 and noting the position of the cartridge plunger. As previously described, the device may be actuated to deliver each bolus of medicament preferably by the concurrent depression of the actuator buttons 834 and 836.

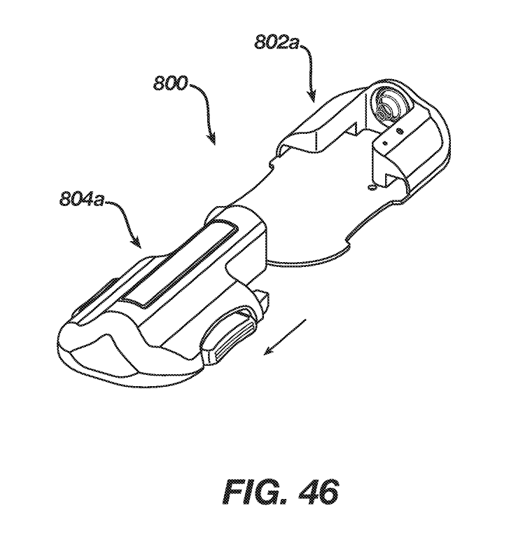

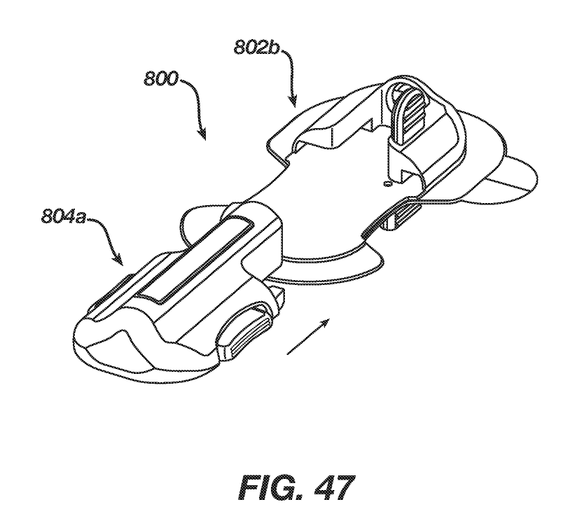

FIGS. 46 and 47 illustrate the convenience and flexibility afforded by this embodiment of the present invention. Generally, a cannula should not be left in a subcutaneous position within a patient for more than about three days. Otherwise, the infusion site could become infected. Hence, it is possible that when it is time to remove a cannula, there may still be medicament remaining in the cartridge reservoir in use. In this event, the entire device may be removed from the patient but not discarded in its entirety. The pump unit may be reused. As a result, as seen in FIG. 46, the pump unit in use 802a may be removed from the old base 802a. Then, as shown in FIG. 47, the old pump unit 804a may be releasably joined with a new base 802b. Thereafter, the new assemblage of the new base 802b and reused pump unit 804a may be deployed as previously described.

Of course, should the cartridge reservoir of a pump unit become empty before it is time to remove the base and cannula, the spent pump unit may be simply removed from the base and replaced with a new pump unit having a new cartridge reservoir. Still further, it is possible to reuse a pump unit. Hence, if a base need not be removed but a cartridge reservoir becomes empty, the pump unit may simply be removed from the base, the spent cartridge reservoir may be removed from the pump unit, a new cartridge reservoir may be inserted into the pump unit, and the reused pump unit equipped with the new cartridge reservoir may be releasably joined with the base.

While particular embodiments of the present invention have been shown and described, modifications may be made. It is therefore intended in the appended claims to cover all such changes and modifications which fall within the true spirit and scope of the invention as defined by those claims.

1.-11. (canceled)

12. A drug infusion system comprising:
a base having a cannula well arranged to receive a cannula that conducts the drug to beneath a wearer's skin, the base further including a base surface arranged to attach to the skin of the wearer, the base including the cannula well and arranged to dispose a cannula to extend from the base surface to beneath the wearer's skin, the base further including an inlet arranged to receive the drug, a conduit that conducts the drug from the inlet to the cannula well, and a first self sealing penetrable barrier moveable with respect to the inlet; and
a reusable drug dispenser removably attachable to the base and having a second self sealing penetrable barrier, a reservoir arranged to hold the drug, and a pump that pumps the drug to the second self sealing penetrable barrier, the first and second self sealing penetrable barriers being arranged to engage each other and be penetrated by the inlet of the base when the reusable dispenser is attached to the base to form an antiseptic connection between the cannula well and the reservoir.

13. The system of claim 12, further comprising a latch assembly that releasably holds the reusable dispenser on the base.

14. The system of claim 12, further comprising a cannula set including a receiving pike and the cannula, the receiving pike being arranged to be received within the cannula well in fluid communication with the conduit, whereby a fluid connection is formed from the cannula through the conduit to the reservoir.

15. The system of claim 12, wherein the base includes a guide that guides the reusable dispenser into attachment on the base.

16. The system of claim 12, wherein the base is coextensive with the reusable dispenser when the reusable dispenser is attached to the base.

17. The system of claim 12, wherein the reusable dispenser includes an inlet cavity adjacent the second self sealing penetrable barrier that receives the inlet of the base when the reusable dispenser is attached to the base, the inlet cavity being arranged to receive the drug from the reservoir and provide the drug to the inlet of the base.

18. The system of claim 12, wherein the reusable dispenser includes a pair of actuators operatively associated with the pump for causing the pump to pump the drug from the reservoir to the cannula upon concurrent actuation of the actuators.

19. The system of claim 12, wherein the inlet of the base has a distal end that penetrates the first and second self sealing penetrable barriers when the reusable dispenser is attached to the base, wherein the first self sealing penetrable barrier is moveable with respect to the inlet of the base and wherein the base further includes a biasing element that urges the first self sealing penetrable barrier against the second self sealing penetrable barrier when the reusable dispenser is attached to the base.

20. The system of claim 12, wherein the reservoir comprises a separate reservoir unit that holds the drug to be delivered, the reservoir unit being engageable with the dispenser on the base.

21. The system of claim 20, further comprising at least one latch that maintains the dispenser and reservoir unit in engagement, the at least one latch comprising a latching projection and a projection receiving slot.

22. The system of claim 12, further comprising a vent immediately adjacent the second self sealing penetrable barrier.
